(19) United States
(12) Patent Application Publication
(10) Pub. No.: US 2026/0153527 A1
(43) Pub. Date: Jun. 4, 2026

(54) COMPLEX SAMPLE PRETREATMENT APPARATUS AND COMPLEX SAMPLE PRETREATMENT METHOD

(71) Applicant: MICRO DIGITAL CO., LTD, Seongnam-si, Gyeonggi-do (KR)

(72) Inventors: Kyung Nam KIM, Gwangju-si, Gyeonggi-do (KR); Se Hak LIM, Gunpo-si, Gyeonggi-do (KR); Jong In RYU, Yongin-si, Gyeonggi-do (KR); Tae Hyun KIM, Seongnam-si, Gyeonggi-do (KR)

(21) Appl. No.: 19/099,885

(22) PCT Filed: Nov. 2, 2023
(86) PCT No.: PCT/KR2023/017357

§ 371 Date: Jan. 30, 2025

(30) Foreign Application Priority Data
Nov. 11, 2022 (KR) 10-2022-0150846

(51) Int. Cl. G01N 35/00; G01N 1/28

(52) U.S. Cl. CPC G01N 35/0099; G01N 1/28; G01N 35/0098; G01N 2035/00356

(57) ABSTRACT

The present invention relates to a complex sample pretreatment apparatus that enables extraction of a DNA or RNA component from a complex sample, such as excretion, which is a mixture of liquid and solid phases, through a series of pretreatment processes. The apparatus may comprise: a housing; a cartridge seating device which is installed inside the housing and onto which a cartridge is seated; a spin drive device which is installed in the housing, is coupled to an object to be spun which is mounted on the cartridge or mounted on the cartridge mounting device, and rotates the object to be spun; and a pump drive device which is installed in the housing, is coupled to a dispensing tip mounted on the cartridge or on the cartridge mounting device, and dispenses a sample or reagent by using the dispensing tip.

### TECHNICAL FIELD

The present invention relates to a complex sample pretreatment apparatus, and more specifically, to a complex sample pretreatment apparatus and a complex sample pretreatment method that enable extraction of a DNA or RNA component from a complex sample, such as excretion, which is a mixture of liquid and solid phases, through a series of pretreatment processes.

Additionally, the present invention was carried out as part of a research project supported by the Ministry of Trade, Industry and Energy and the Korea Evaluation Institute of Industrial Technology under the “Bio-Industry Technology Development-Digital Healthcare” program. [Project title: Development of a Non-invasive Complex Sample Pretreatment Automation System for Digital Healthcare, Project number: 20008702, Project serial number: 1415179590]

### BACKGROUND ART

Molecular diagnosis refers to a diagnostic method that directly analyzes the genes (DNA or RNA) of a target substance in a sample to identify the presence of infection of disease, base sequence variations, or mutations, enabling early disease diagnosis and efficient treatment.

Recently, molecular diagnostic methods have been widely used in various medical fields, including confirmation of disease infection, genetic testing, and pharmacogenetic testing.

Various detection methods have been developed for the molecular diagnostic methods, with real-time polymerase chain reaction (PCR) becoming widely used due to its speed, convenience, and sensitivity in detection. Real-time PCR typically uses a probe that forms a specific complementary binding with the gene of a target substance, and fluorescence molecules are attached to the probe. In real-time PCR, the wavelength of these fluorescence molecules is analyzed by an analyzing device to qualitatively/quantitatively analyze the target gene.

In the molecular diagnostic methods, through real-time PCR, the target substance collected on a swab or a collection part is subjected to pretreatment before analysis, and the pretreated substance, i.e., a buffer solution, is analyzed. In conventional techniques, to collect specimens using swabs, a target specimen, i.e., sample, may be collected in various ways, for example, by using nasal swabs, nasopharyngeal swabs, throat swabs, and the like.

However, the molecular diagnostic methods using swabs can mainly collect specimens in a liquid state and thus are unable to test complex samples where liquid and solid are mixed, such as feces. Particularly, to collect the specimen on the swab, it is necessary to insert the swab deep into the area, such as the nasal cavity, nasopharynx, or throat, which puts strain on the body, causing severe discomfort or strong aversion in a test subject, or if the specimen collection process is not smooth, it results in decreased precision of test results. Additionally, there are many side effects, such as inducing coughing in the test subject, potentially spreading bacteria or viruses to the collector.

### DETAILED DESCRIPTION OF THE INVENTION

### Technical Problem

The present invention is to provide a complex sample pretreatment apparatus and a complex same pretreatment method that enable molecular diagnostic testing of a complex sample comprising a mixture of liquid and solid phases, such as the faces of a test subject, thereby preventing physical discomfort or resistance in the test subject, facilitating sample collection to significantly enhance the precision of test results, and minimizing the spread of bacteria or viruses during the sample collection process. However, these objects are merely illustrative, and the scope of the present invention is not limited thereto.

### Technical Solution

According to an aspect of the present invention, a complex sample pretreatment apparatus may include: a housing; a cartridge seating device which is installed inside the housing and onto which a cartridge is seated; a spin drive device which is installed in the housing, is coupled to an object to be spun which is mounted on the cartridge or mounted on the cartridge seating device, and rotates the object to be spun; and a pump drive device which is installed in the housing, is coupled to a dispensing tip mounted on the cartridge or on the cartridge mounting device, and dispenses a sample or reagent by using the dispensing tip.

In addition, according to the present invention, the cartridge seating device may include a cartridge mounting board on which the cartridge is mounted; and a mounting board moving device configured to move the cartridge mounting board in a first direction so that the cartridge mounted on the cartridge mounting board is positioned at a location corresponding to the spin drive device or the pump drive device.

In addition, according to the present invention, the mounting board moving device may include a driven pulley freely rotatably installed on one side of the housing or frame; a drive pulley freely rotatably installed on the other side of the housing or frame; a mounting board drive motor configured to rotate the drive pulley; and a belt fixed at one side to the cartridge mounting board, wound between the driven pulley and the drive pulley, and configured to move along a track.

In addition, according to the present invention, the cartridge seating device may further include a heater device installed on the cartridge mounting board and configured to heat the cartridge.

In addition, according to the present invention, the heater device may include a heating block installed on the cartridge mounting board and thermally contacting with at least a portion of the cartridge; a heater configured to heat the heating block; a heat-resistant sponge installed between the heater and the cartridge mounting board; and a fastener configured to elastically couple the heating block to the cartridge mounting board using the heat-resistant sponge.

In addition, according to the present invention, the spin drive device may include a spin head drive device which comprises a spin head to be coupled to a complex sample collector or a stirring tip body of the cartridge and moves the spin head up and down or spins the spin head; and a magnetic bar drive device which comprises a magnetic bar, which is formed to pass through the spin head and is inserted into the stirring tip body, when the stirring tip body is coupled to the spin head, to generate magnetic force to allow magnetic beads in the cartridge to stir a sample, and moves the magnetic bar up and down.

In addition, according to the present invention, the spin head drive device may include the spin head formed in a shape corresponding to the complex sample collector or the stirring tip body; a spin head moving board configured to freely support the spin head; a spin rotation motor installed on the spin head moving board and connected to a rotation axis of the spin head to spin the spin head; a head lifting and lowering threaded rod which passes through the spin head moving board to allow the spin head moving board to move up and down and is freely installed on the housing or a frame installed in the housing; and a head lifting and lowering motor configured to rotate the head lifting and lowering threaded rod.

In addition, according to the present invention, the magnetic bar drive device may include the magnetic bar configured to pass through the spin head and allow the magnetic bead to move; a magnetic bar moving board configured to support the magnetic bar; a drive nut freely rotatably installed on the magnetic bar moving board, screwed with the head lifting and lowering threaded rod, and capable of screw-driven vertical movement; a nut rotation motor which is installed on the magnetic bar moving board to enable the magnetic bar moving board to move up and down and is connected to the drive nut to rotate the drive nut; and a moving board linear guide configured to guide lifting and lowering paths of the magnetic bar moving board and the spin head moving board to allow the lifting and lowering path of the magnetic bar moving board to coincide with the lifting and lowering path of the spin head moving board.

In addition, according to the present invention, the pump drive device may include a pump head formed in a shape corresponding to the dispensing tip; a dispensing pump connected to the pump head; a pump moving board configured to support the dispensing pump; a pump lifting and lowering threaded rod which passes through the pump moving board to allow the pump moving board to move up and down and is freely rotatably installed on the housing or a frame installed in the housing; a pump lifting and lowering motor configured to rotate the pump lifting and lowering threaded rod; and a dispensing tip removal device which interferes with the spin drive device and removes the dispensing tip from the pump head through the spin drive device.

In addition, according to the present invention, the dispensing tip removal device may include a dispensing tip removal board which comprises a side projection formed to interfere with the spin drive device, is shaped to surround the pump head to separate the dispensing tip from the pump head as the dispensing tip removal board moves downward, and is formed to be movable up and down on the pump moving board; an elastic spring configured to provide an elastic restoring force in an upward direction of the dispensing tip removal board; and a removal board linear guide configured to guide lifting and lowering paths of the pump moving board and the dispensing tip removal board to allow the lifting and lowering path of the pump moving board to coincide with the lifting and lowering path of the dispensing tip removal board.

Additionally, according to the present invention, the complex sample pretreatment apparatus may further include at least one of the following: an air conditioning device which is installed in the housing and includes at least one of a ventilation fan, a deodorization device, an air purification device, or a filter, or a combination thereof; and an ultraviolet sterilization device installed in the housing and configured to sterilize the cartridge or the cartridge seating device after pretreatment.

Also, according to the present invention, the cartridge may include: a cartridge body; a complex sample collector for collecting a complex sample to provide same to the cartridge body; a buffer solution receiving unit formed in the cartridge body and configured to accommodate a buffer solution therein and provide a space in which, after a collecting unit of the complex sample collector is immersed in the buffer solution, the collecting unit is rotated by a spin head of a robot device such that the complex sample is mixed with the buffer solution or mixed beads included in the buffer solution; and a complex sample collector disposal unit formed in the cartridge body and configured to temporarily store the complex sample collector to discard same after the complex sample is provided to the buffer solution receiving unit.

In addition, according to the present invention, the complex sample collector may include: a collector body having an overall shape of a pipe and a hollow portion formed therein; a collection plunger installed to move up and down in the hollow portion, exposing the collecting unit when the button unit is pressed, and sealing the collected complex sample by retracting the collecting unit into the collector body when the button unit is raised after collecting the complex sample; an isolation protrusion unit formed on a side surface of the collection plunger and spaced apart from the collector body to facilitate rotation of the collection plunger when the button unit descends; a forced-engagement protrusion unit formed on a side surface of the collection plunger to be forcibly engaged with the collector body such that the collecting unit is sealed when the button unit rises; and a locking flange unit installed at an entrance of the collector body and shaped to correspond to a height-lock slot formed on an upper surface of the buffer solution receiving unit such that the spin head of the robot device is inserted in the direction of the height-lock slot and locked into the height-lock slot.

In addition, according to the present invention, the cartridge may further include: a first tip receiving unit formed in the cartridge body and configured to accommodate the first tip such that a first tip is attached to a pump head of the robot device; a filter unit receiving unit formed in the cartridge body and configured to accommodate a filter unit such that the pump head of the robot device attaches the filter unit to the first tip and primarily filters the buffer solution accommodated in the buffer solution receiving unit through the filter unit; and a second tip receiving unit formed in the cartridge body and configured to accommodate a second tip such that the pump head of the robot device uses the first tip to aspirate the buffer solution and attaches the second tip to the first tip.

Additionally, according to the present invention, the filter unit may include a filter unit body forcibly engaged with the first tip; and a flange unit formed on a bottom of the filter unit body and having a plurality of filter holes formed to primarily filter the buffer solution.

In addition, according to the present invention, the first tip may include a first forced-engaging unit formed to forcibly engage with the filter unit, the buffer solution receiving unit may include a second forced-engaging step unit formed to forcibly engage with the filter unit, and a first engagement strength of the first forced-engaging unit may be lower than a second engagement strength of the second forced-engaging step unit such that, after the primary filtering by the filter unit, the filter unit remains in the buffer solution receiving unit, and instead, the first tip is easily separated from the filter unit.

Additionally, according to the present invention, the second tip may further include: a second tip body forcibly engaged with the first tip; and a secondary filter installed inside the second tip body and including a mesh filter or a membrane filter.

In addition, according to the present invention, the cartridge may further include: a sample solution receiving unit formed in the cartridge body and configured to accommodate a sample solution that the pump head of the robot device dispenses while secondarily filtering the buffer solution using the second tip; a first precision dispensing tip receiving unit formed in the cartridge body and configured to accommodate a first precision dispensing tip such that the pump head of the robot device is coupled with the first precision dispensing tip to aspirate the sample solution; a first washing chamber unit formed in the cartridge body and configured to accommodate a first washing solution and magnetic beads included in the first washing solution such that the sample solution aspirated by the pump head of the robot device is mixed with the first washing solution and the magnetic beads to retain only DNA components on the magnetic beads; a stirring tip receiving unit formed in the cartridge body and configured to accommodate a stirring tip such that the spin head of the robot device is coupled with the stirring tip and stirs the magnetic beads in the first washing chamber unit with the first washing solution while rotating or moving up and down; and an Nth washing chamber unit (N is a natural number greater than or equal to 2) formed in the cartridge body and configured to accommodate an Nth washing solution such that, as the stirring tip of the spin head of the robot device rotates or moves up and down, the magnetic beads are mixed with the Nth washing solution, allowing only DNA components to remain.

In addition, according to the present invention, the stirring tip may include: a rotatable stirring tip body having an overall hollow shape and coupled with the spin head; and a magnetic bar installed inside the stirring tip body and magnetically attaching the magnetic beads to the stirring tip body while moving up and down.

Additionally, according to the present invention, the cartridge may include: an elution chamber unit formed in the cartridge body and configured to accommodate an elution solution such that the DNA components remaining on the magnetic bead are separated into the elution solution as the stirring tip of the spin head of the robot device rotates or moves up and down; a second precision dispensing tip receiving unit formed in the cartridge body and configured to accommodate a second precision dispensing tip such that the pump head of the robot device attaches to the second precision dispensing tip; and a pipetting chamber unit formed in the cartridge body and configured to accommodate the elution solution dispensed such that the pump head of the robot device aspirates the elution solution using the second precision dispensing tip and dispenses the aspirated elution solution into the pipetting chamber unit.

### Advantageous Effects

According to some embodiments of the present invention as described above, it is possible to conduct molecular diagnostic testing on a complex sample, such as the feces of a test subject, which comprises a mixture of liquid and solid phases, thereby preventing physical discomfort or resistance in a test subject, facilitating sample collection to significantly enhance the precision of test results, and minimizing the spread of bacteria or viruses during the sample collection process. Additionally, using a complex sample pretreatment apparatus that automates these processes may enable an operation of spinning a complex sample collector with a spin head coupled to the complex sample collector, as well as an intricate and three-dimensional operation involving inserting a magnetic bar into a stirring tip body and moving it upward and downward to allow the spin head to rotate the stirring tip body while simultaneously stirring magnetic beads, optimization of the design, such as reducing the number of threaded rods and simplifying components using a drive nut, is possible, thereby lowering the production cost, and various functionalities and performances may be achieved, such as automatically removing a dispensing tip using neighboring components such as a spin head moving board or the like. However, the scope of the present invention is not limited by the above effects.

### BRIEF DESCRIPTION OF DRAWINGS

FIG. 1 is an exterior perspective view of a cartridge for extraction of a complex sample according to some embodiments of the present invention.

FIG. 2 is a plan view of the cartridge for extraction of a complex sample of FIG. 1.

FIG. 3 is a cross-sectional view of the cartridge for extraction of a complex sample of FIG. 1.

FIGS. 4 to 22 are cross-sectional views of stages of a sample extraction process of the cartridge for extraction of a complex sample of FIG. 1.

FIG. 23 is a flowchart illustrating a complex sample extracting method according to some embodiments of the present invention.

FIG. 24 is a flowchart illustrating in more detail operation (b) of the complex sample extracting method of FIG. 23.

FIG. 25 is a flowchart illustrating in more detail operation (c) of the complex sample extracting method of FIG. 23.

FIG. 26 is an exterior perspective view of a complex sample pretreatment apparatus according to some embodiments of the present invention.

FIG. 27 is a rear view of the complex sample pretreatment apparatus shown in FIG. 26.

FIG. 28 is a perspective view showing an open state of a door of the complex sample pretreatment apparatus shown in FIG. 26.

FIG. 29 is a perspective view showing an internal state of the complex sample pretreatment apparatus shown in FIG. 26.

FIG. 30 is a perspective view showing an internal state of the complex sample pretreatment apparatus of FIG. 26, with a frame removed.

FIG. 31 is a perspective view of a cartridge seating device of the complex sample pretreatment apparatus shown in FIG. 30.

FIG. 32 is a cross-sectional view of a heater device of the cartridge seating device shown in FIG. 31.

FIG. 33 is a perspective view of a spin drive device of the complex sample pretreatment apparatus shown in FIG. 30.

FIG. 34 is an exploded perspective view of a spin drive device of the complex sample pretreatment apparatus shown in FIG. 33.

FIG. 35 is a partial cut-away perspective view of the spin drive device of the complex sample pretreatment apparatus shown in FIG. 33.

FIG. 36 is a perspective view of a pump drive device of the complex sample pretreatment apparatus shown in FIG. 30.

FIG. 37 is an exploded perspective view of the pump drive device of the complex sample pretreatment apparatus shown in FIG. 36.

FIGS. 38 to 40 are front views sequentially illustrating the operation process of a dispensing tip removal device in the complex sample pretreatment apparatus.

### MODE FOR INVENTION

Hereinafter, various preferred embodiments of the present invention will be described in detail with reference to the accompanying drawings.

The embodiments of the present invention are provided for more fully describing the present invention to those skilled in the art, and the embodiments below may be modified in various forms, and the scope of the present invention is not limited to the embodiments below. Rather, these embodiments are provided so that this disclosure will be thorough and complete, and will fully convey the scope of the invention to those skilled in the art. Also, thickness or sizes of layers in the drawings are exaggerated for convenience of explanation and clarity.

First, a cartridge for extraction of a complex sample that can be used in a complex sample pretreatment apparatus will be described in detail.

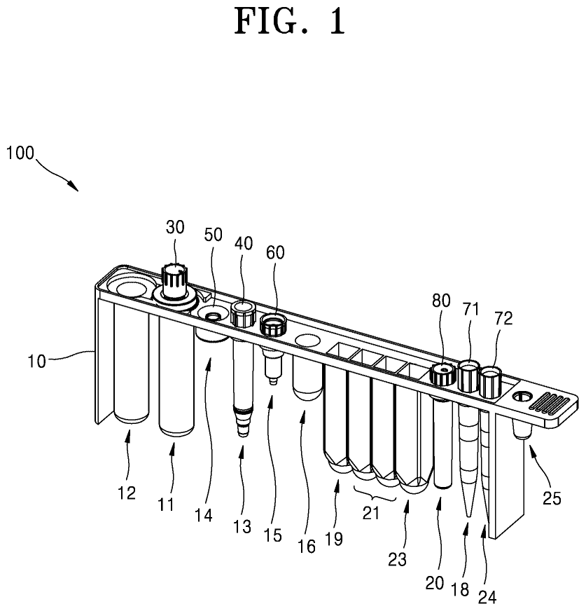

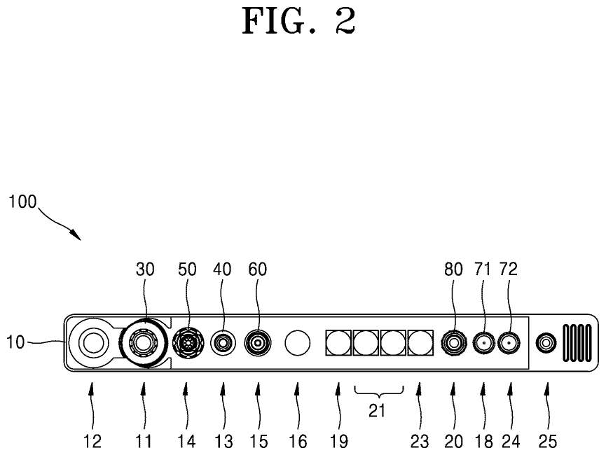

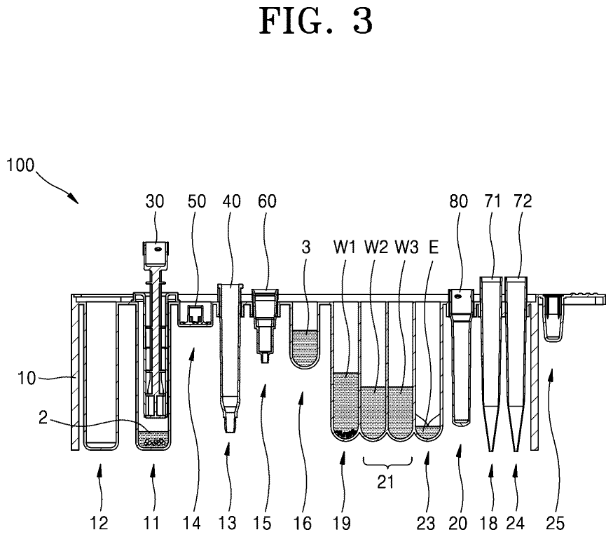

FIG. 1 is an exterior perspective view of a cartridge 100 for extraction of a complex sample according to some embodiments of the present invention, FIG. 2 is a plan view of the cartridge 100 for extraction of a complex sample of FIG. 1, and FIG. 3 is a cross-sectional view of the cartridge 100 for extraction of a complex sample of FIG. 1.

First, as shown in FIGS. 1 to 3, the cartridge 100 for extraction of a complex sample according to some embodiments of the present invention may largely include a cartridge body 10, a buffer solution receiving unit 11 formed in the cartridge body 10, a complex sample collector disposal unit 12, a first tip receiving unit 13, a filter unit receiving unit 14, a second tip receiving unit 15, a sample solution receiving unit 16, a first precision dispensing tip receiving unit 18, a first washing chamber unit 19, a stirring tip receiving unit 20, an Nth washing chamber unit 21, an elution chamber unit 23, a second precision dispensing tip receiving unit 24, and a pipetting chamber unit 25. Further, the cartridge 100 for extraction of a complex sample may further include a complex sample collector 30 configured to collect a complex sample and provide it to the cartridge body 10.

For example, as shown in FIGS. 1 to 3, the cartridge body 10, which may be detachably mounted inside inspection equipment (not shown) installed at an inspection site or at the point of care, may be an integrated structure made of synthetic resin material or metal material with sufficient strength and durability to support the buffer solution receiving unit 11, the complex sample collector disposal unit 12, the first tip receiving unit 13, the filter unit receiving unit 14, the second tip receiving unit 15, the sample solution receiving unit 16, the first precision dispensing tip receiving unit 18, the first washing chamber unit 19, the stirring tip receiving unit 20, the Nth washing chamber unit 21, the elution chamber unit 23, the second precision dispensing tip receiving unit 24, and the pipetting chamber unit 25 described above.

However, the shape, type, material, design, and the like of the cartridge body 10 are not limited thereto, and they may be modified or changed as needed depending on the specifications of the equipment where the cartridge is to be mounted, the inspection environment, or the required specifications.

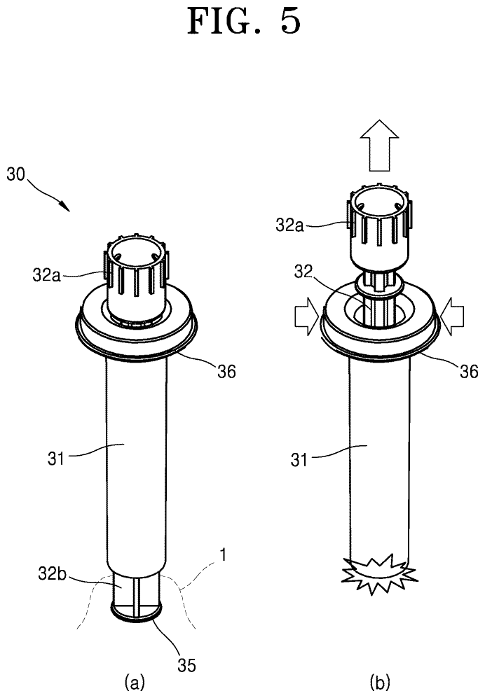

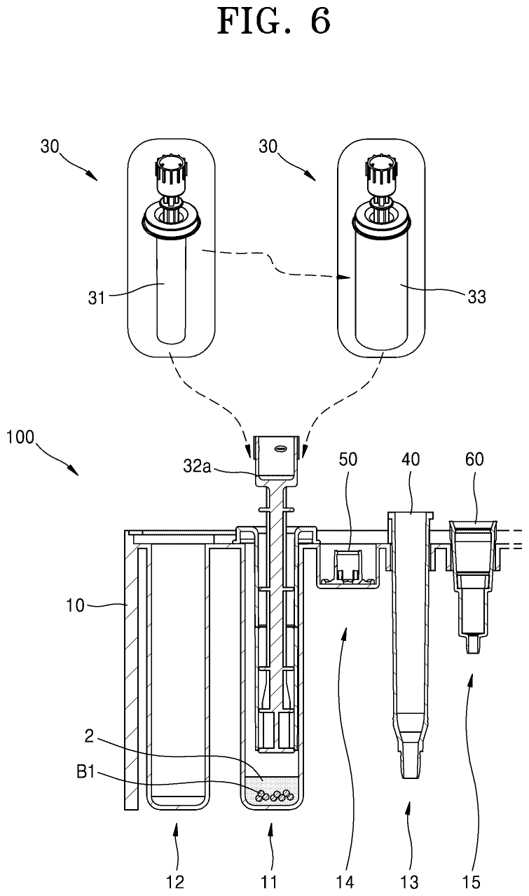

More specifically, as shown in FIGS. 1 to 3, for example, the buffer solution receiving unit 11, formed in the cartridge body 10, may accommodate a buffer solution 2 therein, provide a space where, after a collecting unit 32b (shown in FIG. 5) of the complex sample collector 30, which has collected a complex sample 1 (shown in FIG. 5) such as feces, is immersed in the buffer solution 2, the collecting unit 32b is rotated by a spin head SH of a robot device, allowing the complex sample 1 to be mixed with the buffer solution 2 or mixed beads B1 (shown in FIG. 6) contained in the buffer solution 2, and accommodate both the buffer solution 2 and the complex sample collector 30.

Additionally, for example, as shown in FIGS. 1 to 3, the complex sample collector disposal unit 12, formed in the cartridge body 10, may serve as a part that temporarily stores the complex sample collector 30 to discard the same after the complex sample 1 is provided to the buffer solution receiving unit 11.

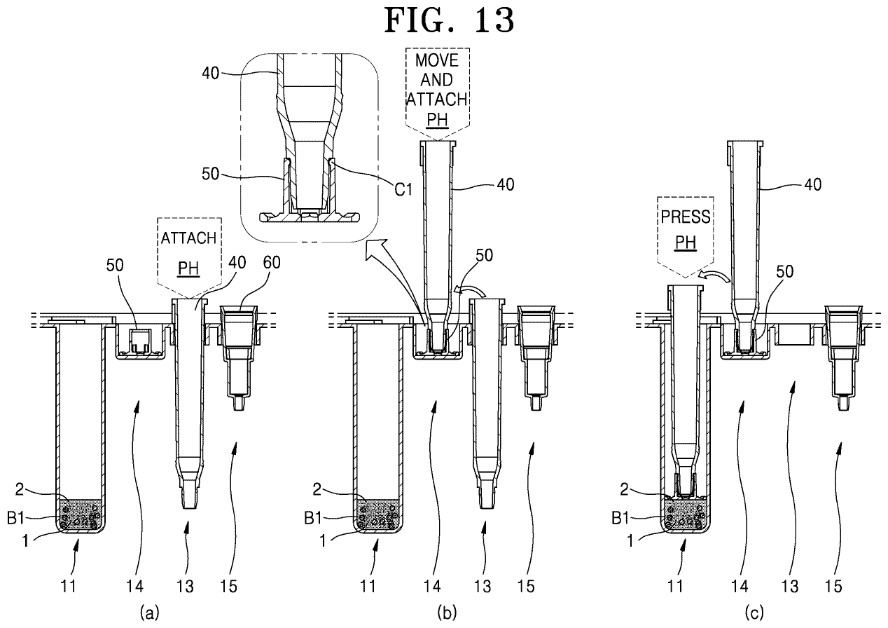

In addition, for example, as shown in FIGS. 1 to 3, the first tip receiving unit 13, formed in the cartridge body 10, may serve as a part that accommodates the first tip 40 such that a pump head PH (shown in FIG. 13) of the robot device attaches the first tip 40 thereto.

Also, for example, as shown in FIGS. 1 to 3, the filter unit receiving unit 14, formed in the cartridge body 10, may serve as a part that accommodates the filter unit 50 such that the pump head PH of the robot device attaches the filter unit 50 to the first tip 40, allowing the buffer solution 2, for example, fibers or debris contained in the buffer solution 2, within the buffer solution receiving unit 11 to be filtered by the filter unit 50. These fibers or debris may originate from the complex sample 1.

In addition, for example, as shown in FIGS. 1 to 3, the second tip receiving unit 15, formed in the cartridge body 10, may serve as a part that accommodates the second tip 60 such that the pump head PH of the robot device uses the first tip 40 to aspirate the buffer solution 2 and attaches the second tip 60 to the first tip 40. Specifically, while the first tip 40 aspirates and holds the buffer solution 2, the second tip 60 may be attached to one end of the first tip 40.

Moreover, for example, as shown in FIGS. 1 to 3, the sample solution receiving unit 16, formed in the cartridge body 10, may serve as a part that accommodates the sample solution 3 that is dispensed by the pump head PH of the robot device while secondarily filtering the buffer solution 2 using the second tip 60.

The sample solution 3 may refer to a substance formed after the complex sample 1 and the buffer solution 2 are mixed and subsequently filtered. Therefore, the sample solution 3 may refer to a substance that includes the buffer solution 2, the liquid substance of the complex sample 1, and a liquid substance formed by dissolving the solid substance of the complex sample 1 in the buffer solution 2.

After dispensing the sample solution 3 into the sample solution receiving unit 16, the buffer solution receiving unit 11, the first tip receiving unit 13, or the second tip receiving unit 15 temporarily stores the engaged first and second tips 40 and 60, functioning as a first and second tip disposal unit that discards the engaged first and second tips 40 and 60. Additionally, the first and second tip disposal unit may be provided as a separate configuration.

In addition, for example, as shown in FIGS. 1 to 3, the first precision dispensing tip receiving unit 18, formed in the cartridge body 10, may serve as a part that accommodates the first precision dispensing tip 71 such that the pump head PH of the robot device is coupled with the first precision dispensing tip 71 and aspirates the sample solution 3.

Also, for example, as shown in FIGS. 1 to 3, the first washing chamber unit 19, formed in the cartridge body 10, may serve as a part that accommodates a first washing solution W1 and magnetic beads B2 such that the sample solution 3 aspirated by the pump head PH of the robot device is mixed with the first washing solution W1 and the magnetic beads B2 included in the first washing solution W1, allowing only DNA components to remain on the magnetic beads B2.

The magnetic beads B2 may consist of a material with magnetic properties, for example, metal. The magnetic beads B2 may be composed of various materials, shapes, and forms to which the DNA components can be attached physically, chemically, biologically, or the like.

In addition, for example, as shown in FIGS. 1 to 3, the stirring tip receiving unit 20, formed in the cartridge body 10, may serve as a part that accommodates the stirring tip 80 such that the spin head SH of the robot device is coupled with the stirring tip 80 and stirs the magnetic beads B2 with the first washing solution W1 in the first washing chamber unit 19 while rotating or moving up and down.

Additionally, the stirring tip receiving unit 20 may function as a stirring tip disposal unit that temporarily stores the stirring tip 80 for subsequent disposal of the stirring tip 80. Also, the stirring tip disposal unit may be provided as a separate configuration.

Moreover, for example, as shown in FIGS. 1 to 3, the Nth washing chamber unit 21, formed in the cartridge body 10, may serve as a part that accommodates an Nth washing solution WN (where N is a natural number greater than or equal to 2) such that as the stirring tip 80 of the spin head SH of the robot device rotates or moves up and down, the magnetic beads B2 magnetically attached to and transferred by the stirring tip 80 are mixed with the Nth washing solution WN, allowing only the DNA components to remain.

Additionally, for example, as shown in FIGS. 1 to 3, the elution chamber unit 23, formed in the cartridge body 10, may serve as a part that accommodates an elution solution E such that the DNA components remaining on the magnetic beads B2, which are magnetically attached to and transferred by the stirring tip 80 of the spin head SH of the robot device as the stirring tip 80 rotates or moves up and down, are separated into the elution solution E.

Also, for example, as shown in FIGS. 1 to 3, the second precision dispensing tip receiving unit 24, formed in the cartridge body 10, may serve as a part that accommodates the second precision dispensing tip 72 such that the pump head PH of the robot device is coupled with the second precision dispensing tip 72.

Moreover, for example, as shown in FIGS. 1 to 3, the pipetting chamber unit 25, formed in the cartridge body 10, may serve as a part that accommodates the elution solution E dispensed such that the pump head PH of the robot device aspirates the elution solution E using the second precision dispensing tip 72 and dispenses the aspirated elution solution E into the pipetting chamber unit 25.

Here, for example, as shown in FIGS. 1 to 3, according to the order of the pretreatment processes for extraction of a complex sample, the buffer solution receiving unit 11, the complex sample collector disposal unit 12, the first tip receiving unit 13, the filter unit receiving unit 14, the second tip receiving unit 15, the sample solution receiving unit 16, the first and second tip disposal unit 17, the first precision dispensing tip receiving unit 18, the first washing chamber unit 19, the stirring tip receiving unit 20, the Nth washing chamber unit 21, the elution chamber unit 23, the second precision dispensing tip receiving unit 24, and the pipetting chamber unit 25 may be arranged in a line in this order, from the front end to the rear end of the upper surface of the cartridge body 10.

However, this order is not necessarily limited to the drawings, and various rearrangements are possible as needed.

Therefore, as shown in FIGS. 1 to 3, the movement distances of the spin head SH and pump head PH of the robot device installed in the aforementioned inspection equipment may be minimized. In other words, while the spin head SH and pump head PH intermittently move from the front end to the rear end of the cartridge body 10, a series of sample extraction pretreatment processes of extracting the complex sample 1 from the complex sample collector 30 and dispersing the same into the elution solution E in the pipetting chamber section 25 may be sufficiently performed sequentially within only a single cartridge 100 for extraction of a complex sample according to some embodiments of the present invention.

Therefore, to apply a molecular diagnostic method to a complex sample such as feces, a series of pretreatment processes of collecting the complex sample 1 using the complex sample collector 30 and extracting the same with the elution solution E are automated and integrated in a cartridge format, so that the efficiency of sample extraction may be increased, the time and cost involved in sample extraction may be significantly reduced, and precise, uniform, and highly reliable sample pretreatment processes may be conducted regardless of the operator's skill level or working environment. Also, sample contamination or leakage during the process may be fundamentally prevented, and the application to an on-site rapid diagnostic kit is possible, enabling quick achievement of test results, on-site, within several minutes or hours.

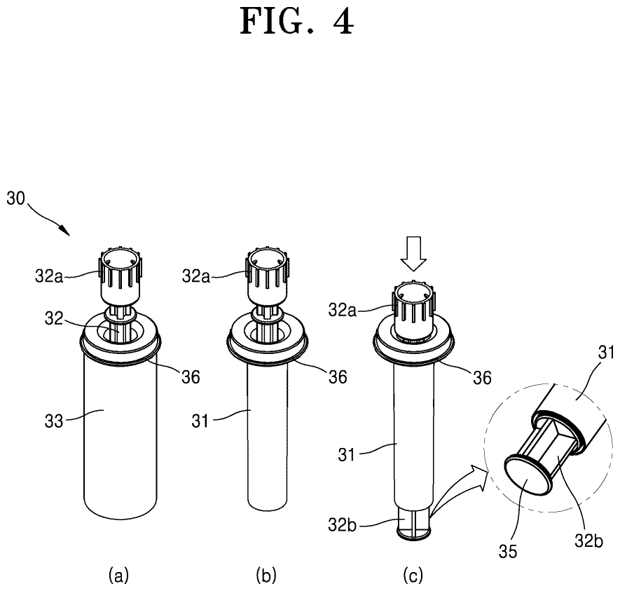

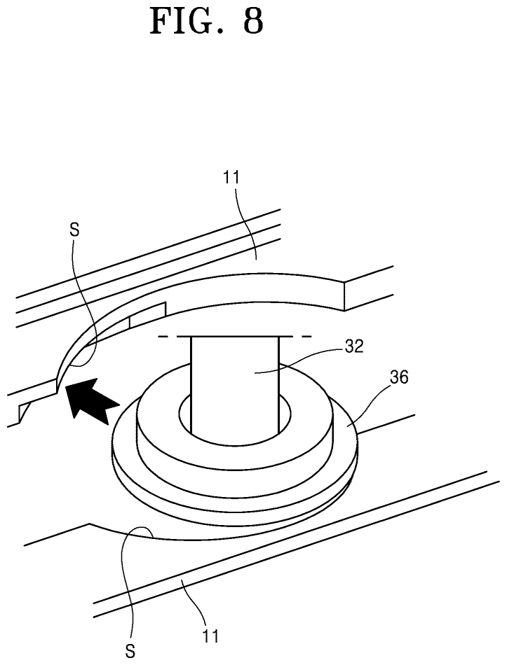

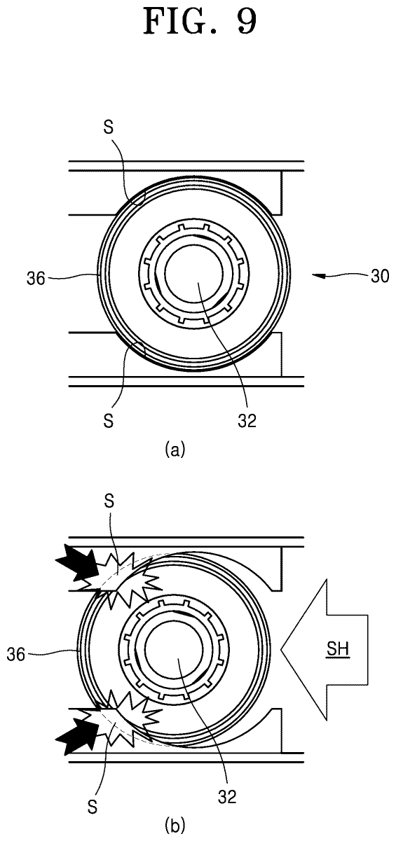

FIGS. 4 to 22 are cross-sectional views of stages of a sample extraction process of the cartridge 100 for extraction of a complex sample of FIG. 1.

As shown in FIGS. 4 to 22, a more detailed description of the extraction process of the cartridge 100 for extraction of a complex sample according to some embodiments of the present invention is provided. Firstly, as shown in FIG. 4, using the complex sample collector 30, a complex sample 1 such as feces may be collected and stored.

Here, as shown in FIG. 4, the complex sample collector 30 may include a collector body 31 having the overall shape of a pipe and a hollow portion formed therein and a collection plunger 32 installed to move up and down in the hollow portion, exposing the collecting unit 32b when a button unit 32a is pressed, and sealing the collected complex sample 1 by retracting the collecting unit 32b into the collector body 31 when the button unit 32a is raised. Also, the complex sample collector 30 may further include a storage container 32 that surrounds and protects at least a portion of the collector body 33 and the collection plunger 31.

Therefore, as shown in (a) of FIG. 4, a collector may store the complex sample collector 30 using the storage container 33 during regular times. Also, during collection preparation, the collector may separate the storage container 33 as shown in (b) of FIG. 4, and expose the collecting unit 32b externally by pressing the button unit 32a as shown in (c) of FIG. 4.

Subsequently, as shown in (a) of FIG. 5, the collector may insert the exposed collecting unit 32b into the complex sample 1 and evenly apply the sample to the inner surface of the collecting unit 32b. Following this, as shown in (b) of FIG. 5 the collector may lift the button unit 32a to seal the collecting unit 32b inside the collector body 31.

Next, as shown in FIG. 6, the collector may insert the collector body 31 directly into the buffer solution receiving unit 11 or temporarily store it in the storage container 33. Then, after separating the storage container 33, the collector may insert the collector body 31 into the buffer solution receiving unit 11 to immerse the collecting unit 32b of the complex sample collector 30, which has collected the complex sample 1, such as feces, in the buffer solution 2.

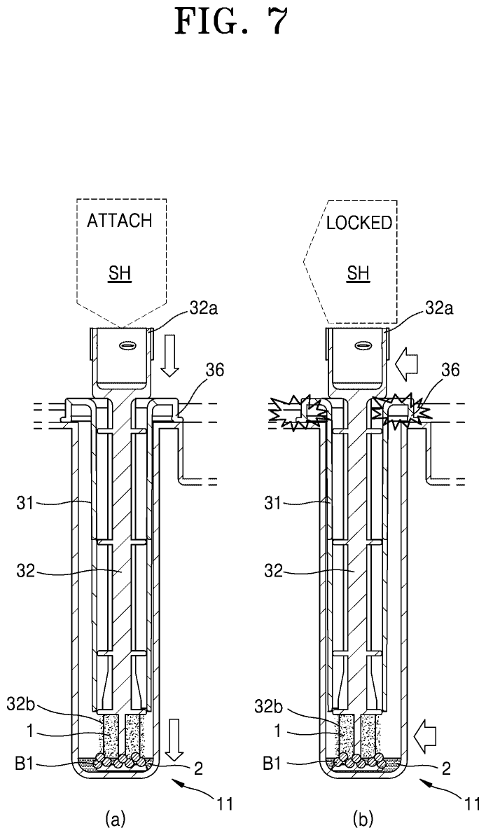

In this case, as shown in FIGS. 7 to 9, the complex sample collector 30 may further include a locking flange unit 36 installed at the entrance of the collector body 31 and shaped to correspond to a height-lock slot S formed on the upper surface of the buffer solution receiving unit 11 such that the spin head SH of the robot device can be inserted in the direction of the height-lock slot S and locked into the height-lock slot S. The complex sample collector 30 may be inserted and locked into the height-lock slot S using the locking flange unit 36.

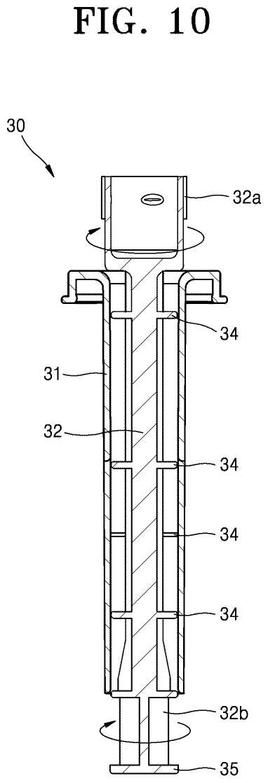

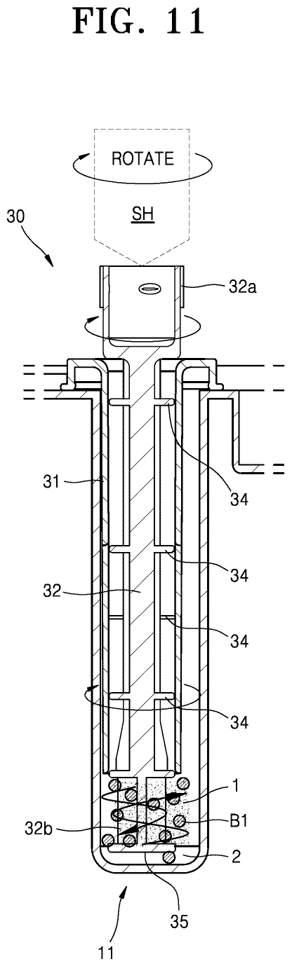

Next, as shown in FIGS. 10 and 11, the complex sample collector 30 may further include an isolation protrusion unit 34 formed on the side surface of the collection plunger 32 and spaced apart from the collector body 31 to facilitate the rotation of the collection plunger 32 when the button unit 32a descends, and a forced-engagement protrusion unit 35 formed on the side surface of the collection plunger 32 to be forcibly engaged with the collector body 31 such that the collecting unit 32b is sealed when the button unit 32a rises. When the button unit 32a descends, the spin head SH of the robot device may freely rotate the collecting unit 32b, allowing the complex sample 1 to be mixed with the buffer solution 2 or the mixed beads B1 included in the buffer solution 2.

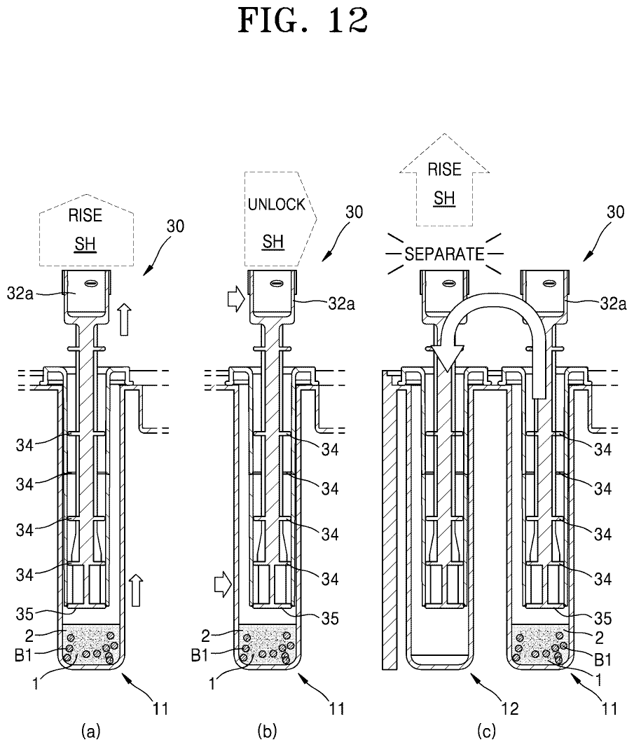

Subsequently, as shown in FIG. (a) of FIG. 12, the spin head SH of the robot device raises the button unit 32a to seal the collecting unit 32b. Then, as shown in (b) of FIG. 12, the position of the spin head SH is moved to the right to release the height lock, and finally, as shown in (c) of FIG. 12, the complex sample collector 30 may be temporarily stored in the complex sample collector disposal unit 12 for subsequent disposal of the complex sample collector 30.

Next, as shown in (a) of FIG. 13, the pump head PH of the robot device is equipped with the first tip 40, and as shown in (b) of FIG. 13, the filter unit 50 may be attached to the first tip 40.

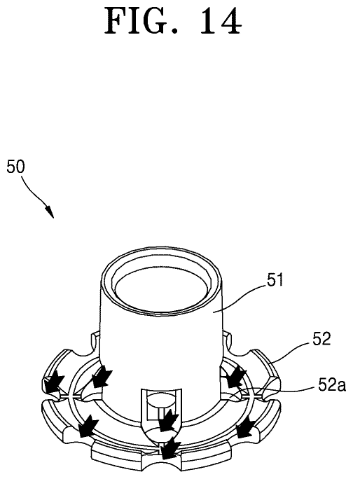

Here, as shown in FIG. 14, the filter unit 50 may include a filter unit body 51 that is forcibly engaged with the first tip 40, and a flange unit 52 formed at the bottom of the filter unit body 51 and having multiple filter holes 52a formed to primarily filter, for example, fibers or debris contained in the buffer solution 2.

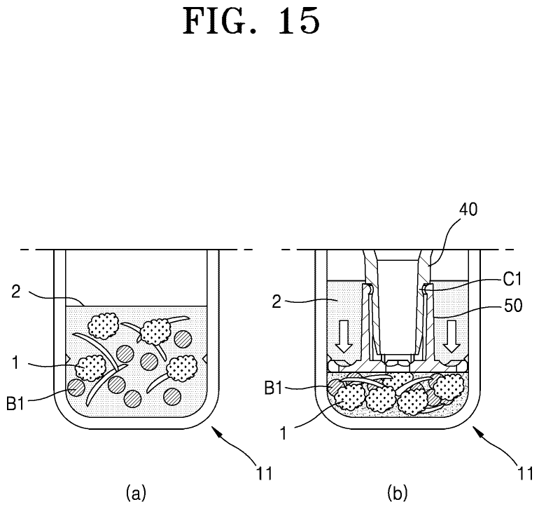

Subsequently, as shown in (a) of FIG. 15, relatively large debris, such as fibers or debris, in the buffer solution 2 inside the buffer solution receiving unit 11 may be pressed through the filter unit 50 as shown in (b) of FIG. 15, allowing the fibers or debris to be primarily filtered, thereby positioning the buffer solution 2 in the upper part of the buffer solution receiving unit 11. Consequently, the liquid substance or solid substance of the complex sample 1 may be dissolved in the buffer solution 2 and positioned in the upper part by passing through the filter unit 50.

Here, as shown in (b) of FIG. 15, the first tip 40 may include a first forced-engaging unit C1 formed to forcibly engage with the filter unit 50, and the buffer solution receiving unit 11 may include a second forced-engaging step unit C2 formed to forcibly engage with the filter unit 50. A first engagement strength of the first forced-engaging unit C1 may be lower than a second engagement strength of the second forced-engaging step unit C2 such that, after the primary filtering by the filter unit 50, the filter unit 50 remains in the buffer solution receiving unit 11, and instead, the first tip 40 may be easily separated from the filter unit 50.

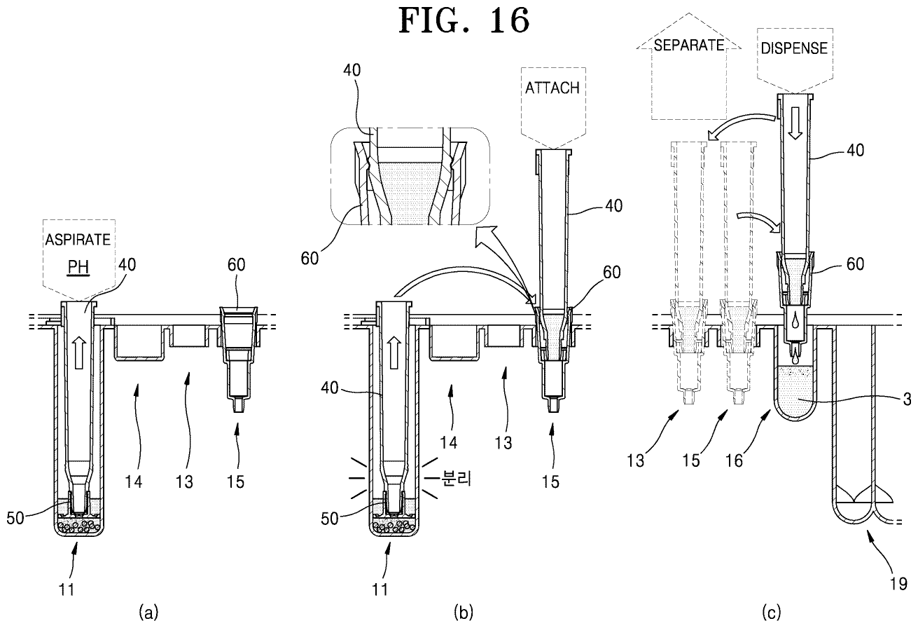

Therefore, as shown in (a) of FIG. 16, the pump head PH of the robot device may aspirate the buffer solution 2 using the first tip 40, and the first tip 40 may rise to separate from the filter unit 50 such that the filter unit 50 remains in the buffer solution receiving unit 11. This separation may be achieved by the difference between the first engagement strength and the second engagement strength. The buffer solution 2 aspirated into the first tip 40 may contain the liquid substance of the complex sample 1 or the solid substance of the complex sample 1 dissolved in the buffer solution 2.

Next, as shown in (b) of FIG. 16, the second tip 60 may be attached to the first tip 40, and as shown in (c) of FIG. 16, the pump head PH of the robot device may use the second tip 60 to dispense the sample solution 3 to the sample solution receiving unit 16 while secondarily filtering the buffer solution 2. After dispensing the sample solution 3, the remaining first tip 40 and the attached second tip 60 may be temporarily stored in the buffer solution receiving unit 11, the first tip receiving unit 13, or the second tip receiving unit 15 for subsequent disposal.

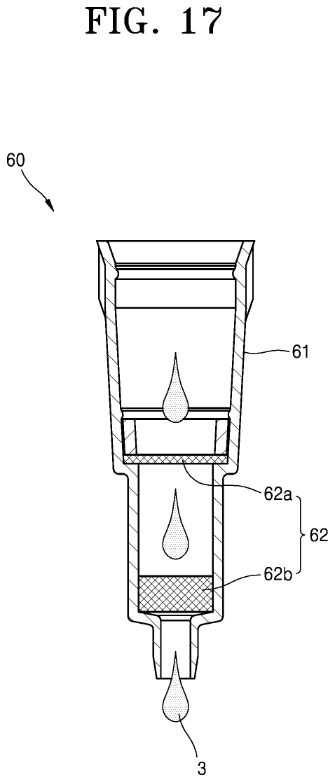

Here, for example, as shown in FIG. 17, the second tip 60 may include a second tip body 61 that is forcibly engaged with the first tip 40, and a secondary filter 62 installed inside the second tip body 61 and including a mesh filter 62a or a membrane filter 62b, and may secondarily filter debris or fibers that may remain in the buffer solution 2.

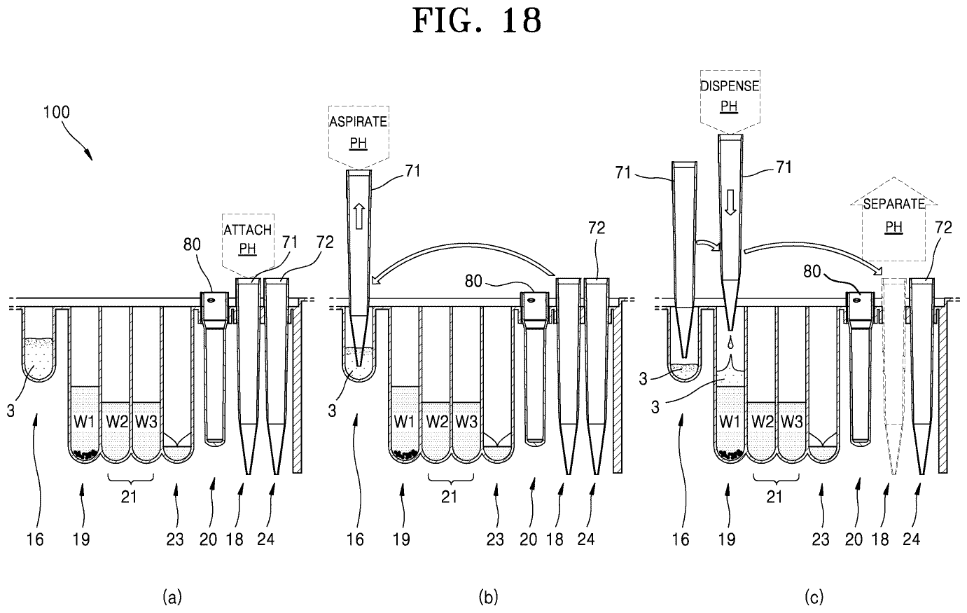

Subsequently, as shown in (a) of FIG. 18, the pump head PH of the robot device may be coupled with the first precision dispensing tip 71, and as shown in (b) of FIG. 18, it may aspirate the sample solution 3 from the sample solution receiving unit 16. Subsequently, as shown in (c) of FIG. 18, the pump head PH of the robot device may dispense the aspirated sample solution 3 into the first washing chamber unit 19 such that the sample solution 3 is mixed with the first washing solution W1 and the magnetic beads B2 included in the first washing solution W1, allowing only the DNA components to remain on the magnetic beads B2.

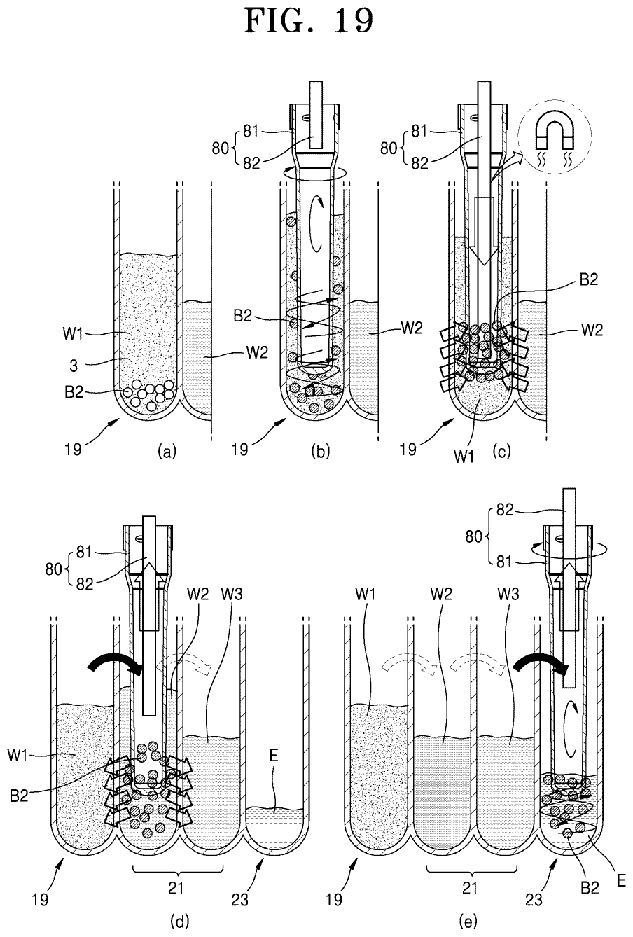

Here, as shown in (a) of FIG. 19, the first washing solution W1 to be mixed with the sample solution 3 may contain a plurality of magnetic beads B2 therein.

Next, as shown in (b) and (c) of FIG. 19, the spin head SH of the robot device may be coupled with the stirring tip 80 and may rotate or move up and down to stir the magnetic beads B2 in the first washing chamber unit 19 with the first washing solution W1. Additionally, the magnetic beads B2 may be magnetically attached to the stirring tip 80.

Subsequently, as shown in (d) and (e) of FIG. 19, in N Nth washing chamber units (N is a natural number greater than or equal to 2), the magnetic beads B2 may be repeatedly stirred N times with the Nth washing solutions WN and W3 as the stirring tip 80 of the spin head SH of the robot device rotates or moves up and down, so that only the DNA component remains.

Here, as shown in FIG. 19, the stirring tip 80 may include a rotatable stirring tip body 81 having an overall hollow shape and coupled with the spin head SH and a magnetic bar 82 installed inside the stirring tip body 81 and magnetically attaching the magnetic beads B2 to the stirring tip body 81 while moving up and down.

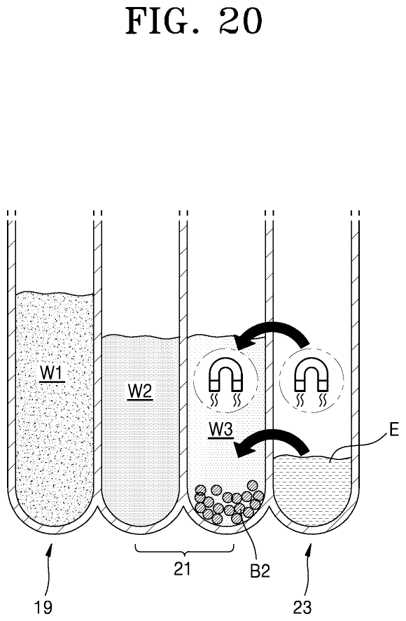

Therefore, as shown in FIG. 20, using the stirring tip 80 allows both the rotation of the stirring tip body 81 and the up-and-down movement of the magnetic bar 82 so that the magnetic beads B2 are shaken and thus can be smoothly stirred. The magnetic beads B2 may be magnetically attached to the stirring tip 80 by the magnetic bar 82 and may be transferred from the first washing chamber unit 19 to the Nth washing chamber unit 21 after stirring, sequentially moved through the Nth washing chamber 21, and ultimately transferred to the elution chamber unit 23. Consequently, the elution solution E may be accommodated in the elution chamber unit 23.

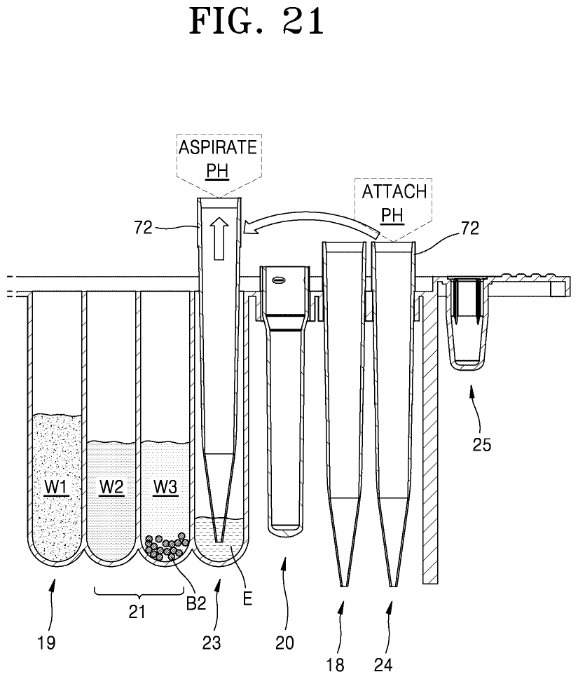

Subsequently, as shown in FIG. 21, the pump head PH of the robot device may be coupled with the second precision dispensing tip 72, and may aspirate the elution solution E accommodated in the elution chamber unit 23 using the second precision dispensing tip 72.

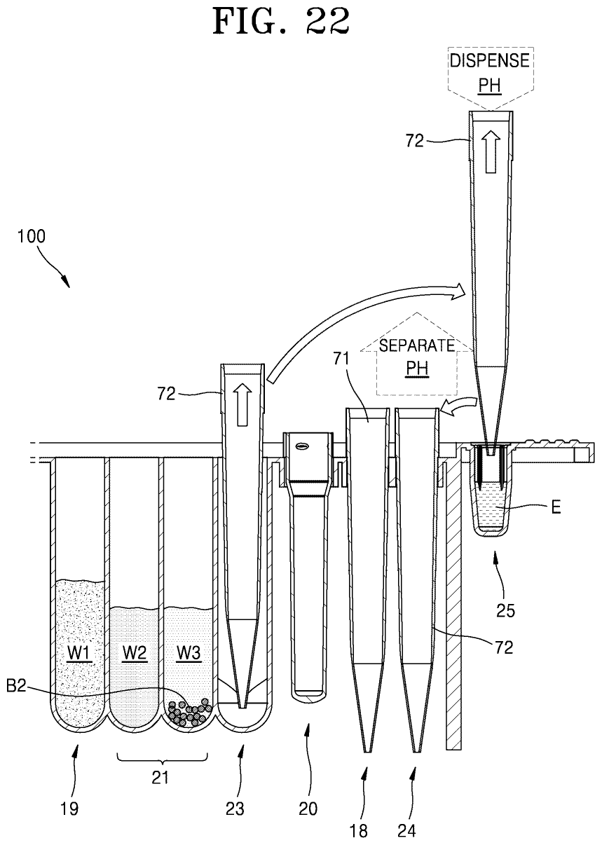

Then, as shown in FIG. 22, the aspirated elution solution E may be dispensed into the pipetting chamber unit 25.

Therefore, an operator may obtain the elution solution E that has undergone the entire pretreatment processes from the pipetting chamber unit 25 and proceed with the analysis process and post-treatment process.

Consequently, molecular diagnostic testing may be performed on the complex sample 1, such as the feces of a test subject, consisting of a mixture of liquid and solid phases, so that physical discomfort or resistance in the test subject can be prevented and sample collection is easy, thereby significantly increasing the precision of test results, while minimizing the spread of bacteria or viruses during the sample collection process.

However, these series of complex sample extraction processes are not limited to the drawings and modifications and changes can be made by those skilled in the art without departing from the technical spirit of the present invention.

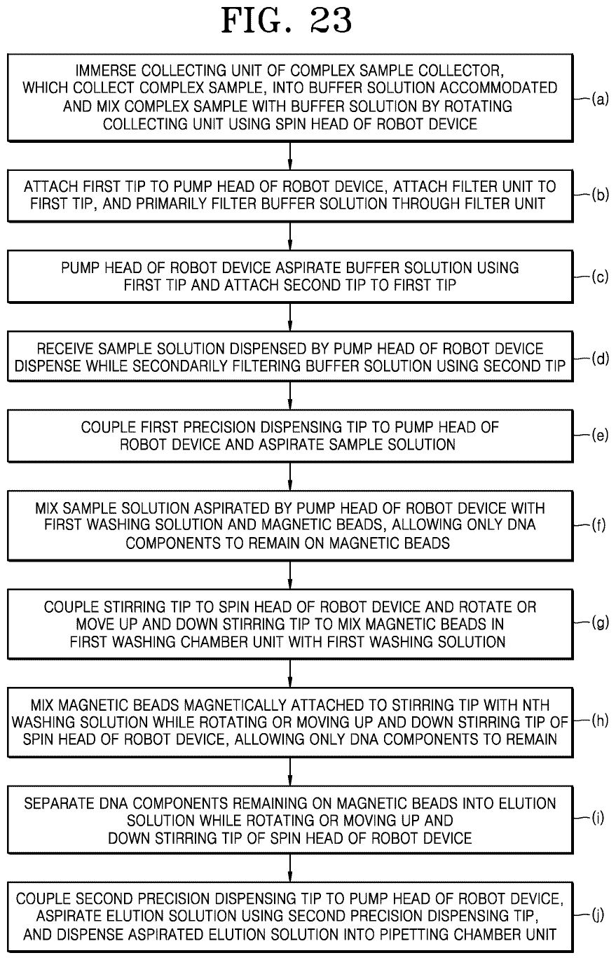

FIG. 23 is a flowchart illustrating a complex sample extracting method according to some embodiments of the present invention.

As shown in FIGS. 1 to 23, a complex sample extracting method according to some embodiments of the present invention may include: (a) immersing the collecting unit 32b of the complex sample collector 30, which has collected the complex sample 1, into the buffer solution 2 accommodated in the buffer solution receiving unit 11 and mixing the complex sample 1 with the buffer solution 2 or the mixed beads B1 contained in the buffer solution 2 by rotating the collecting unit 32b using the spin head SH of the robot device; (b) attaching the first tip 40 to the pump head PH of the robot device, attaching the filter unit 50 to the first tip 40, and primarily filtering the buffer solution 2 accommodated inside the buffer solution receiving unit 11 through the filter unit 50; (c) at the pump head PH of the robot device, aspirating the buffer solution 2 using the first tip 40 and attaching the second tip to the first tip 40; (d) receiving the sample solution 3 that the pump head PH of the robot device dispenses while secondarily filtering the buffer solution 2 contained in the first tip 40 using the second tip 60; (e) coupling the first precision dispensing tip 71 to the pump head PH of the robot device and aspirating the sample solution 3; (f) mixing the sample solution 3 aspirated by the pump head PH of the robot device with the first washing solution W1 accommodated in the first washing chamber unit 19 and the magnetic beads B2 included in the first washing solution W1, allowing only DNA components to remain on the magnetic beads B2; (g) coupling the stirring tip 80 to the spin head SH of the robot device and rotating or moving up and down the stirring tip 80 to mix the magnetic beads B2 in the first washing chamber unit 19 with the first washing solution W1, allowing only the DNA components to remain; (i) separating the DNA components remaining on the magnetic beads B2 into the elution solution E while rotating or moving up and down the stirring tip 80 of the spin head SH of the robot device; and (j) coupling the second precision dispensing tip 72 to the pump head PH of the robot device, aspirating the elution solution E using the second precision dispensing tip 72, and dispensing the aspirated elution solution E into the pipetting chamber unit 25.

The complex sample extracting method may further include, before or after operation (i), (h) mixing the magnetic beads B2 with the Nth washing solution WN (N is a natural number greater than or equal to 2) while rotating or moving up and down the stirring tip 80 of the spin head SH of the robot device, allowing only the DNA components to remain.

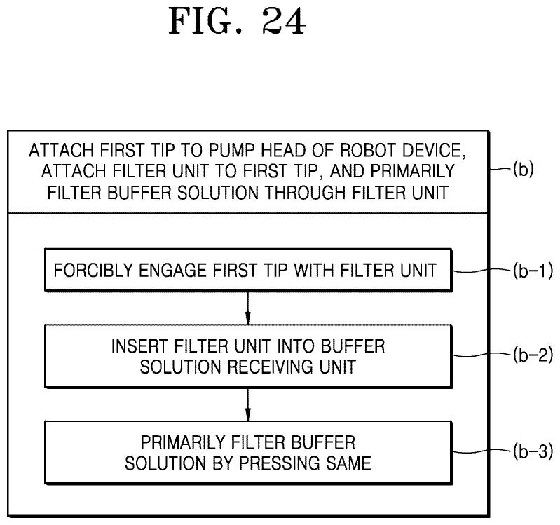

FIG. 24 is a flowchart illustrating in more detail operation (b) of the complex sample extracting method of FIG. 23.

As shown in FIGS. 1 to 24, operation (b) may include: (b-1) forcibly engaging the first tip 40 with the filter unit 50; (b-2) inserting the filter unit 50 into the buffer solution receiving unit 11; and (b-3) primarily filtering the buffer solution 2 by pressing the buffer solution 2 mixed with the complex sample 1 using the filter unit 50.

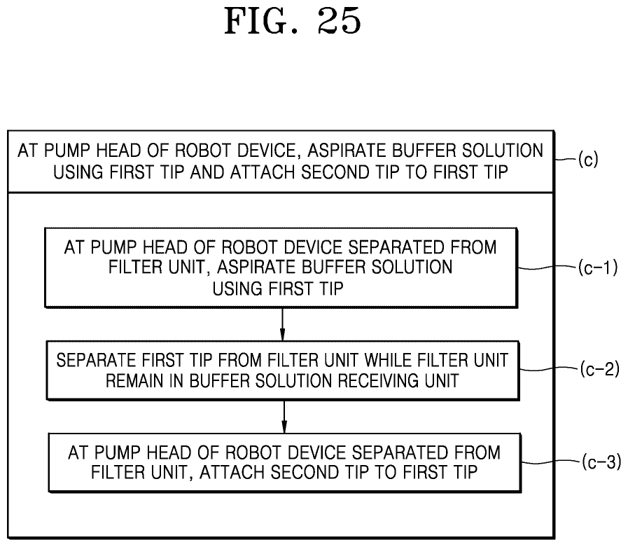

FIG. 25 is a flowchart illustrating in more detail operation (c) of the complex sample extracting method of FIG. 23.

As shown in FIGS. 1 to 25, operation (c) may include: (c-1) at the pump head PH of the robot device separated from the filter unit 50, aspirating the buffer solution 2 using the first tip 40; (c-2) raising the pump head PH of the robot device to separate the first tip 40 from the filter unit 50 while being attached to the pump head PH of the robot device while the filter unit 50 remains in the buffer solution receiving unit 11; and (c-3) at the pump head PH of the robot device separated from the filter unit 50, attaching the second tip 60 to the first tip 40.

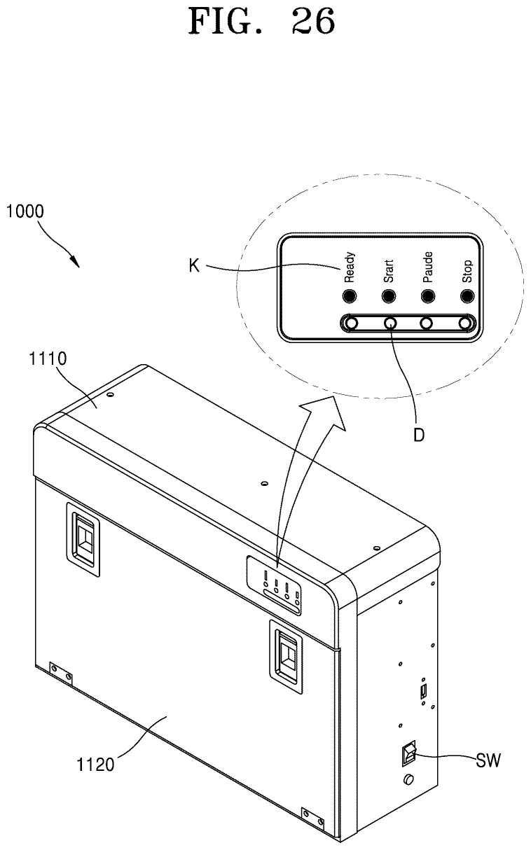

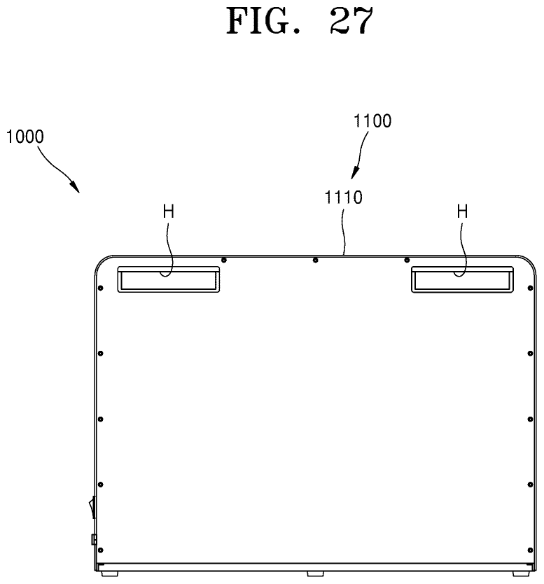

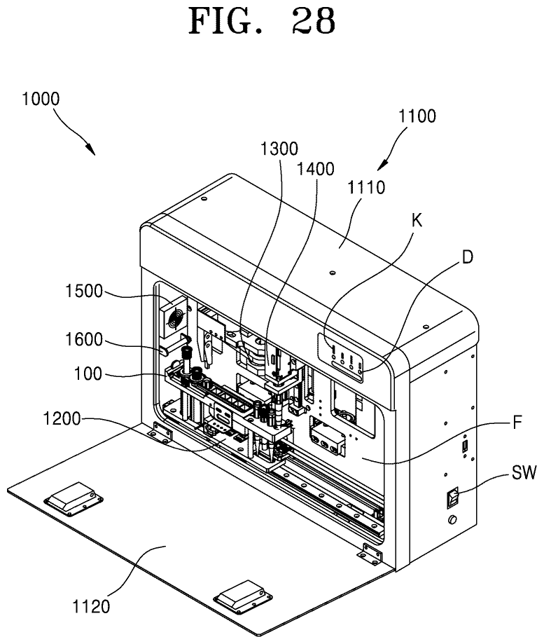

FIG. 26 is an exterior perspective view of a complex sample pretreatment apparatus 1000 according to some embodiments of the present invention, FIG. 27 is a rear view of the complex sample pretreatment apparatus 1000 of FIG. 26, and FIG. 28 is a perspective view showing an open state of a door of the complex sample pretreatment apparatus 1000 of FIG. 26.

First, as shown in FIGS. 26 to 28, the complex sample pretreatment apparatus 1000 according to some embodiments of the present invention may largely include a housing 1100, a cartridge seating device 1200 on which a cartridge 100 is seated, a spin drive device 1300, and a pump drive device 1400.

Here, the cartridge 100 may include the cartridge 100 for extraction of a complex sample described above in FIGS. 1 to 25, which may accommodate a complex mixture of liquid and solid phases, such as feces, sputum, or whole blood.

More specifically, for example, the housing 1100 may be a box-shaped structure with an internal accommodation space, and may include a display unit D on the exterior, such as a lamp, display, or indicator window to show operating status or progress; a command input unit K, such as push buttons for ready, start, pause, or stop; a handle unit H for easily gripping and moving the housing 1100 by hand; a housing body on which a power switch SW or the like is formed; and a door 1120 which is optionally openable and installed on the housing body 1110 to allow loading and unloading of the cartridge 100 into the accommodation space.

However, the housing body 1110 and door 1120 are not necessarily limited to the drawings and may be configured in various forms, taking into account the working environment, specifications, or design considerations.

Additionally, for example, the complex sample pretreatment apparatus 1000 according to some embodiments of the present invention may further include an air conditioning device 1500, which is installed in the housing 1100 to ensure a pleasant environment and prevent odors or contamination and includes a ventilation fan, a deodorization device, air purification device, a filter (e.g., HEPA filter), or the like, and an ultraviolet sterilization device 1600 installed in the housing 1100 to sterilize the internal space, such as the cartridge 100 or the cartridge seating device 1200, either before or after pretreatment.

Therefore, after completing the pretreatment process for a complex sample such as feces, sputum, or whole blood, the air conditioning device 1500, the ultraviolet sterilization device 1600, or the like may be used to maintain cleanliness inside the apparatus, thereby significantly improving the reliability of pretreatment operations for new samples.

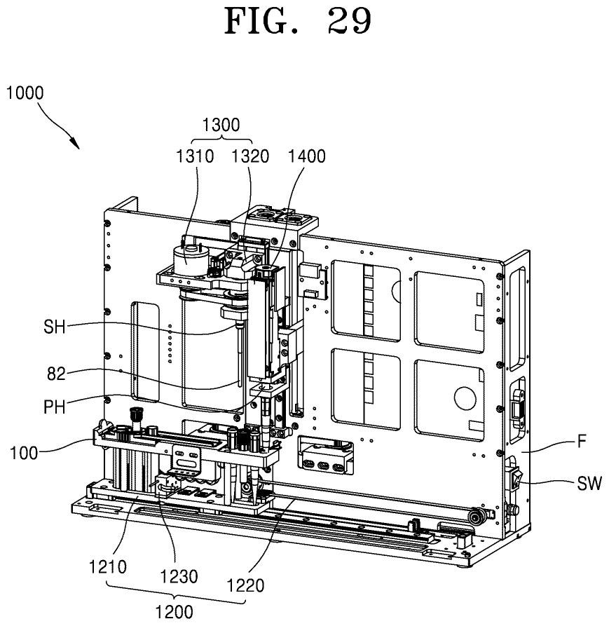

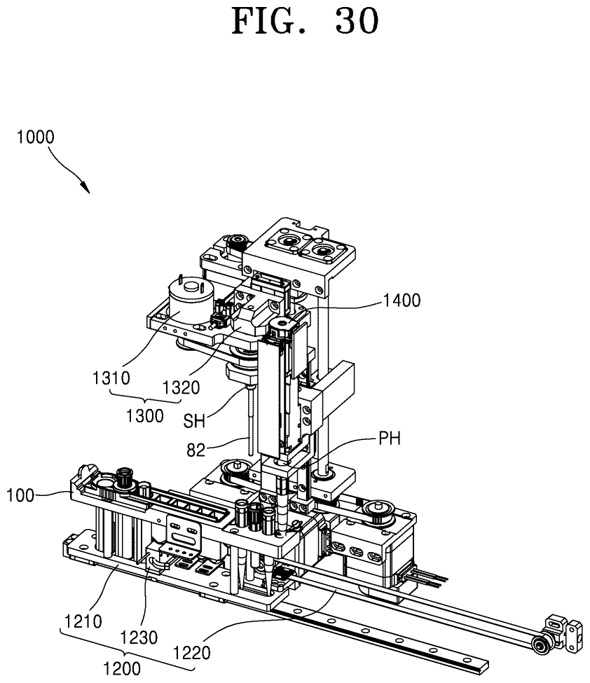

FIG. 29 is a perspective view showing the internal state of the complex sample pretreatment apparatus 1000 of FIG. 26, and FIG. 30 is a perspective view showing the internal state of the complex sample pretreatment apparatus 1000 of FIG. 26 with its frame F removed.

As shown in FIGS. 29 and 30, the cartridge seating device 1200 is a device on which the above-described cartridge 100 is mounted, and may include a cartridge mounting board 1210 where the cartridge 100 is mounted, and a mounting board moving device 1220 that moves the cartridge mounting board 1210 in a first direction (Y-axis direction) so that the cartridge 100 mounted on the cartridge mounting board 1210 can be positioned at a location corresponding to a spin drive device 1300 or a pump drive device 1400.

Therefore, using the mounting board moving device 1220, the cartridge mounting board 1210 may sequentially move the cartridge 100 to a position below the spin drive device 1300 or the pump driving device 1400.

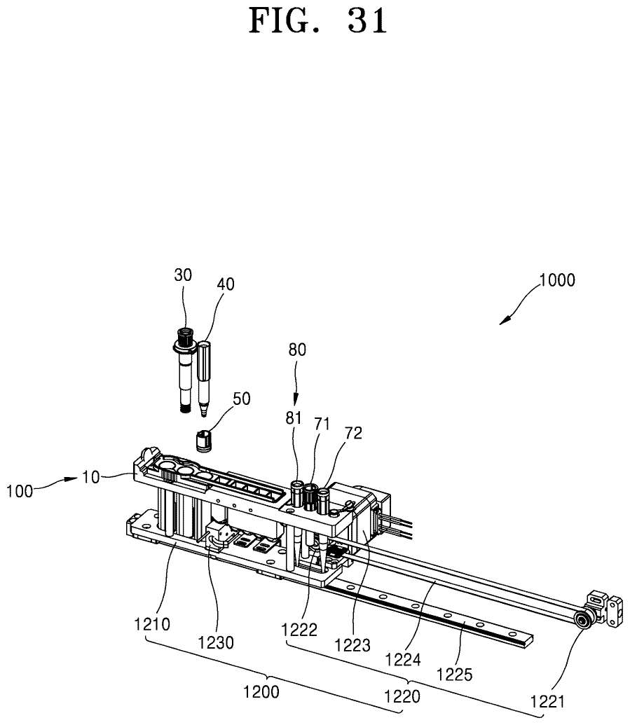

FIG. 31 is a perspective view of a cartridge seating device 1200 of the complex sample pretreatment apparatus 1000 of FIG. 30.

More specifically, as shown in FIG. 31, the mounting board moving device 1220 may include a driven pulley 1221 freely rotatably installed on one side of the housing 1100 or frame F; a drive pulley 1222 freely rotatably installed on the other side of the housing 1100 or frame F; a mounting board drive motor 1223 configured to rotate the drive pulley 1222; a belt 1224 fixed at one end to the cartridge mounting board 1210, wound between the driven pulley 1221 and the drive pulley 1222, and moving along a track; and a mounting board linear guide 1225 configured to guide the linear reciprocating motion path of the cartridge mounting board 1210.

Therefore, when the mounting board drive motor 1223 rotates the drive pulley 1222 in a forward or reverse direction, one side of the belt 1224, wound between the driven pulley 1221 and the drive pulley 1222 and moving along the track, advances or retracts, thereby moving the cartridge mounting board 1210, with the cartridge 100 mounted thereon, forward or backward.

However, in addition to the combination of the belt and pulleys, the mounting board moving device 1220 may also use various other types of moving devices, such as a rack gear and pinion gear combination, a chain and sprocket wheel combination, a moving board and threaded rod combination, a linear motor, or a wire and pulley combination.

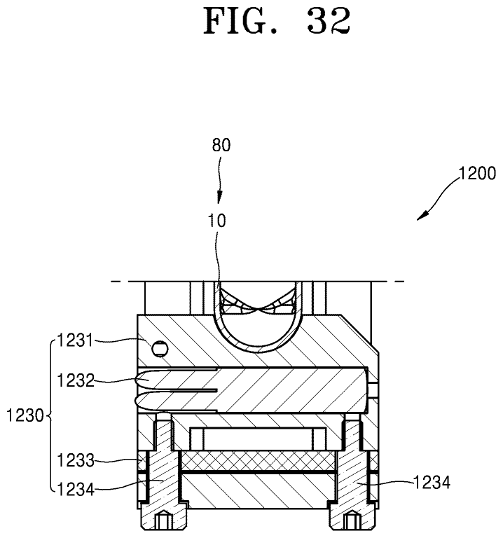

FIG. 32 is a cross-sectional view of a heater device 1230 of the cartridge seating device 1200 shown in FIG. 31.

As shown in FIG. 32, the cartridge seating device 1200 may further include a heater device 1230 installed on the cartridge mounting board 1210 and configured to heat the cartridge 100.

For example, the heater device 1230 may include a heating block 1231 installed on the cartridge mounting board 1210, thermally contacting at least a portion of the cartridge 100 to uniformly heat the sample to a specific temperature; a detachable heater 1232 configured to heat the heating block 1231; a heat-resistant sponge 1233 installed between the heater 1232 and the cartridge mounting board 1210 to prevent the heat of the heater 1232 from transferring to the cartridge mounting board 1210; and fasteners 1234, such as bolts or screws, configured to elastically couple the heating block 1231 to the cartridge mounting board 1210 using the elasticity of the heat-resistant sponge 1233. Therefore, when necessary, it is also possible to heat the sample or solution contained in the cartridge 100 using the heater device 1230. More specifically, for example, the heater device 1230 may maintain the temperature of the internal sample, solution, or liquid medication at 50° C. to 60° C.

In addition, a flexible fixing structure using the heat-resistant sponge 1233 and the fasteners 1234 may allow the cartridge 100 and the heating block 1231 to make contact with an appropriate contact pressure and may ensure stable contact between them at various angles.

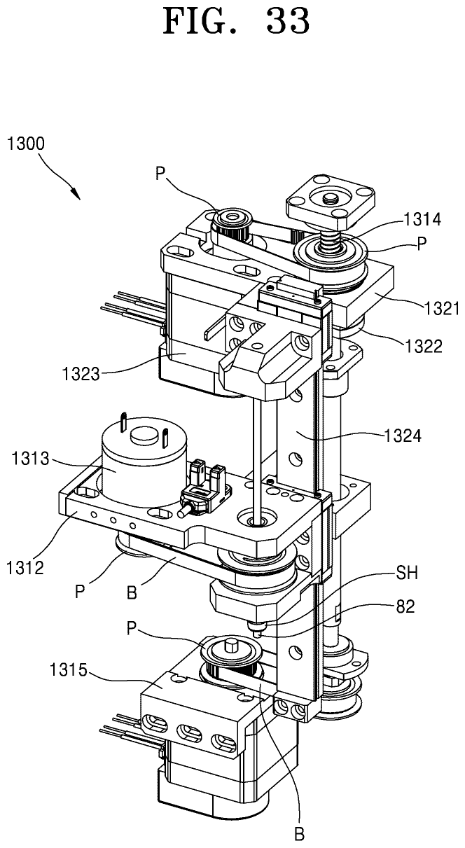

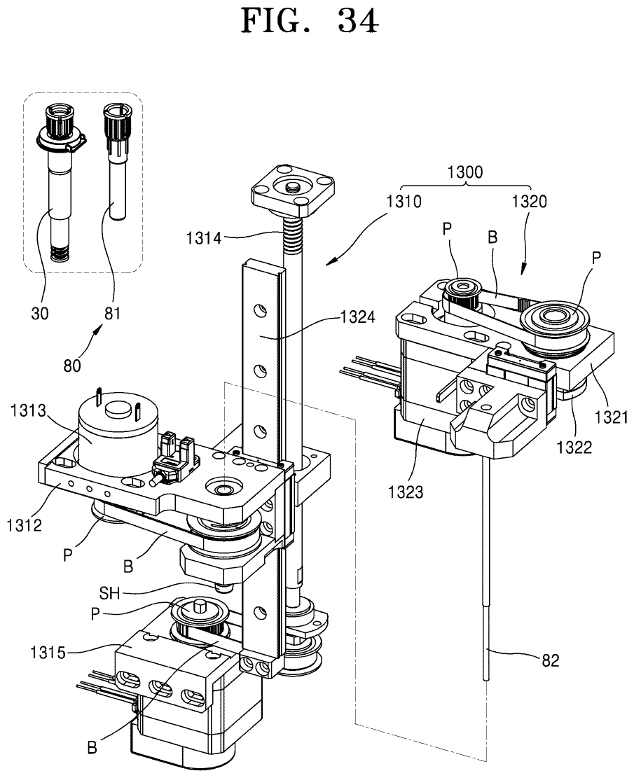

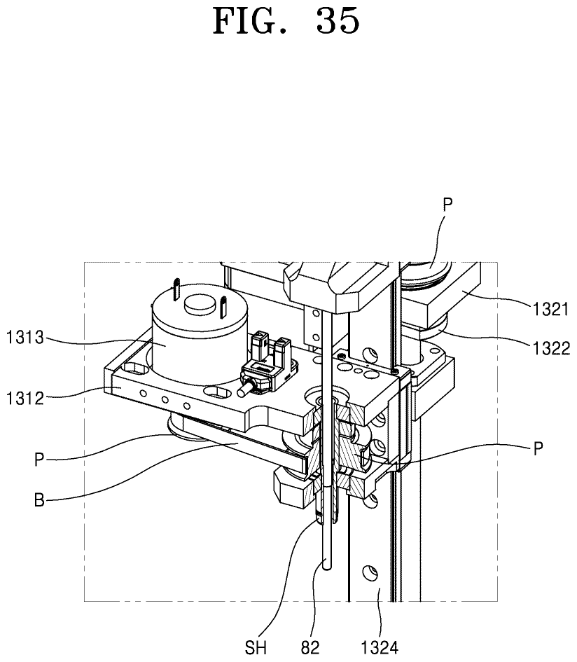

FIG. 33 is a perspective view of the spin drive device 1300 of the complex sample pretreatment apparatus 1000 shown in FIG. 30, FIG. 34 is an exploded perspective view of the spin drive device 1300 of the complex sample pretreatment apparatus 1000 shown in FIG. 33, and FIG. 35 is a partial cut-away perspective view of the spin drive device 1300 of the complex sample pretreatment apparatus 1000 shown in FIG. 33.

As shown in FIGS. 33 to 35, the spin drive device 1300 is installed in the housing 1100 and may be a device coupled to an object to be spun, such as the complex sample collector 30 or the stirring tip body 81 shown in FIGS. 1 to 3, which is mounted on the cartridge 100 or mounted on the cartridge seating device 1200, to rotate the object to be spun.

For example, the spin drive device 1300 may primarily include a spin head drive device 1310 and a magnetic bar drive device 1320.

Here, for example, the spin head drive device 1310 may include a spin head SH that is coupled to the complex sample collector 30 or the stirring tip body 81 of the cartridge 100 and may be a device that moves the spin head SH up and down or spins it.

More specifically, the spin head drive device 1310 may include a spin head SH formed in a shape corresponding to the complex sample collector 30 or the stirring tip body 81; a spin head moving board 1312 configured to freely support the rotation of the spin head SH; a spin rotation motor 1313 installed on the spin head moving board 1312 and connected to the rotation axis of the spin head SH to rotate the spin head SH; a head lifting and lowering threaded rod 1314 that passes through the spin head moving board 1312 to allow the spin head moving board 1312 to move up and down and is freely rotatably installed on the housing 1100 or a frame F installed in the housing 1100; and a head lifting and lowering motor 1315 configured to rotate the head lifting and lowering threaded rod 1314.

Here, for example, the spin rotation motor 1313 may rotate the spin head SH using a combination of a belt B and a pulley P. However, the present invention is not necessarily limited thereto; and a hollow-shaft spin rotation motor 1313 may be directly connected to the spin head SH to rotate it directly.

In addition, for example, the head lifting and lowering motor 1315 may also rotate the head lifting and lowering threaded rod 1314 using a combination of a belt B and a pulley P. However, the present invention is not necessarily limited thereto, the head lifting and lowering motor 1315 may also be directly connected to the head lifting and lowering threaded rod 1314 to rotate it directly.

In this case, such belts B and pulleys P may use a timing belt type for precise control. In addition to these belt and pulley combinations, various other types of movement mechanisms may also be applied, such as a rack gear and pinion gear combination, a chain and sprocket wheel combination, a moving board and threaded rod combination, a linear motor, or a wire and pulley combination.

Additionally, for example, the head lifting and lowering threaded rod 1314 may utilize a ball screw type threaded rod for precise numerical control. However, other various types of lifting and lowering devices, such as a rack gear and pinion gear combination, a chain and sprocket wheel combination, a linear motor, or a wire and pulley combination, may also be applied.

In addition, for example, the magnetic bar drive device 1320 may include a magnetic bar 82, which is formed to pass through the spin head SH and is inserted into the stirring tip body 81, when the stirring tip body 81 is coupled to the spin head SH, to generate magnetic force to allow the magnetic beads B2 in the cartridge 100 shown in FIG. 19 to stir the sample, and may be a device that raises and lowers the magnetic bar 82.

More specifically, for example, the magnetic bar drive device 1320 may include the magnetic bar 82 configured to pass through the spin head SH and enable the magnetic beads B2 shown in FIG. 19 to move; a magnetic bar moving board 1321 configured to support the magnetic bar 82; a drive nut 1322 which is freely rotatably installed on the magnetic bar moving board 1321, screwed with the head lifting and lowering threaded rod 1314, and capable of screw-driven vertical movement; a nut rotation motor 1323 which is installed on the magnetic bar moving board 1321 to enable the magnetic bar moving board 1321 to move up and down and is connected to the drive nut 1322 to rotate the drive nut 1322; and a moving board linear guide 1324 configured to guide the lifting and lowering paths of the magnetic bar moving board 1321 and the spin head moving board 1312, enabling the lifting and lowering path of the magnetic bar moving board 1321 to coincide with the lifting and lowering path of the spin head moving board 1312.

Here, for example, the nut rotation motor 1323 is configured independently of the head lifting and lowering motor 1315, and operates simultaneously or individually without being constrained by the head lifting and lowering motor 1315, and the nut rotation motor 1323 may rotate the drive nut 1322 using a combination of a belt B and a pulley P. However, the present invention is not necessarily limited to this configuration; for instance, other various types of power transmission devices, such as a rack gear and pinion gear combination, a chain and sprocket wheel combination, a moving board and threaded rod combination, a linear motor, or a wire and pulley combination, may also be applied.

Additionally, instead of the nut rotation motor 1323, various types and shapes of motors or actuators, such as hollow motors or hollow rotary actuators that can be directly connected to the drive nut 1322 to rotate it directly, may also be applied.

Therefore, it is possible to perform an intricate

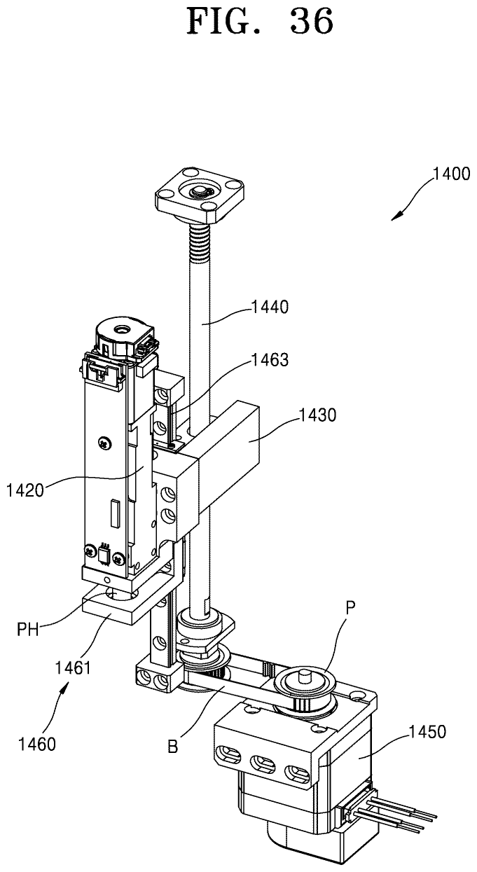

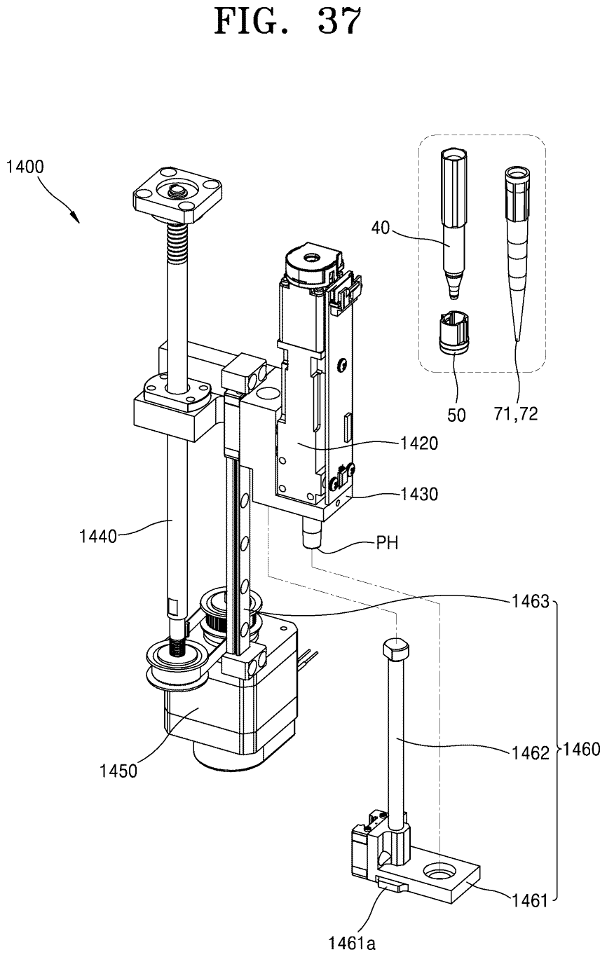

FIG. 36 is a perspective view of the pump drive device 1400 of the complex sample pretreatment apparatus 1000 shown in FIG. 30, and FIG. 37 is an exploded perspective view of the pump drive device 1400 of the complex sample pretreatment apparatus 1000 shown in FIG. 36.

As shown in FIGS. 36 and 37, the pump drive device 1400, installed in the housing 1100, may be a device coupled with the dispensing tip 71 or 72 shown in FIGS. 1 to 3, which is mounted on the cartridge 100 or the cartridge seating device 1200, and may dispense a sample or reagent using the dispensing tip 71 or 72.

For example, the pump drive device 1400 may include a pump head PH formed in a shape corresponding to the dispensing tips 71 and 72; a dispensing pump 1420 with high precision performance connected to the pump head PH; a pump moving board 1430 configured to support the dispensing pump 1420; a pump lifting and lowering threaded rod 1440 configured to pass through the pump moving board 1430 to allow the pump moving board 1430 to move up and down and freely rotatably installed on the housing 1100 or a frame F installed in the housing 1100; and a pump lifting and lowering motor 1450 configured to rotate the pump lifting and lowering threaded rod 1440.

Here, the pump lifting and lowering motor 1450 may rotate the pump lifting and lowering threaded rod 1440 using a combination of a belt B and a pulley P. Alternatively, the pump lifting and lowering motor 1450 may be directly connected to the pump lifting and lowering threaded rod 1440 to rotate it directly.

In addition to the combination of the belt B and pulley P, various other types of moving devices, such as a rack gear and pinion gear combination, a chain and sprocket wheel combination, a moving board and threaded rod combination, a linear motor, or a wire and pulley combination, may also be applied.

Therefore, in the pump drive device 1400, when the pump lifting and lowering motor 1450 rotates the pump lifting and lowering threaded rod 1440 in a forward or reverse direction, the pump moving board 1430 moves up or down along the threads of the threaded rod 1440, allowing the pump head PH to selectively couple with the dispensing tip 71 or 72, and the dispensing pump 1420 may use the dispensing tip 71 or 72 to aspirate or dispense a sample or reagent.

Meanwhile, as shown in FIGS. 36 to 38, the pump drive device 1400 may further include a dispensing tip removal device 1460 which interferes with the spin drive device 1300 and removes the coupled dispensing tip 71 or 72 from the pump head PH after its use through the spin drive device 1300.

For example, the dispensing tip removal device 1460 may include a dispensing tip removal board 1461 which includes a side projection 1461a formed to interfere with the spin drive device 1300, is shaped to surround the pump head PH to separate the dispensing tip 71 or 72 from the pump head PH as the dispensing tip removal board 1461 moves downward, and is formed to be movable up and down on the pump moving board 1430; an elastic spring 1462 configured to provide an elastic restoring force in the upward direction of the dispensing tip removal board 1461; and a removal board linear guide 1463 configured to guide the lifting and lowering paths of the pump moving board 1430 and the dispensing tip removal board 1461 to allow the lifting and lowering path of the pump moving board 1430 to coincide with the lifting and lowering path of the dispensing tip removal board 1461.

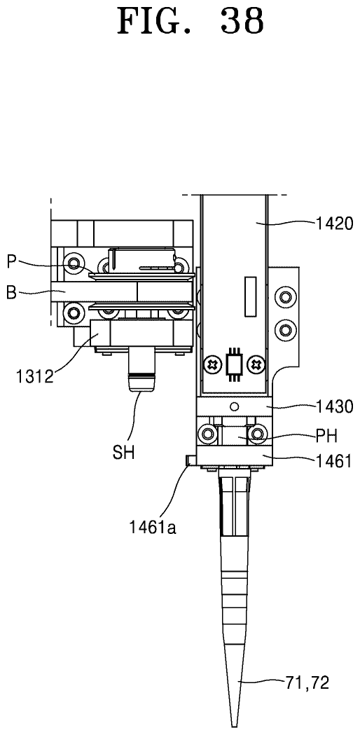

FIGS. 38 to 40 are front views sequentially illustrating the operation process of the dispensing tip removal device 1460 in the complex sample pretreatment apparatus 1000.

As shown in FIGS. 38 to 40, the operation process of the dispensing tip removal device 1460 in the complex sample pretreatment apparatus 1000 is as follow. First, as shown in FIG. 38, when the spin head moving board 1312 of the spin drive device 1300 is in a raised position, the dispensing tip removal board 1461 does not interfere with the spin head moving board 1312, and the pump head PH remains coupled with the dispensing tip 71 or 72 and may aspirate or dispense the sample or solution.

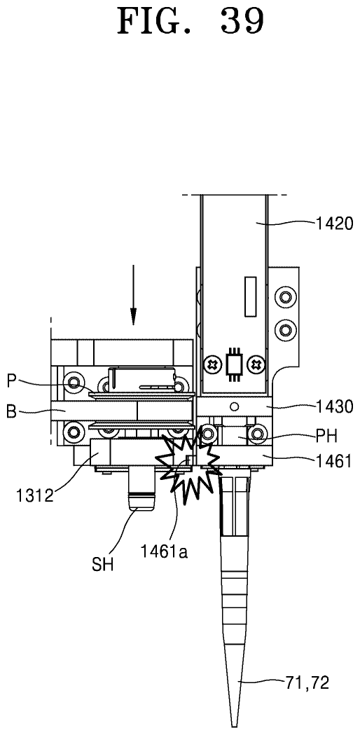

Next, as shown in FIG. 39, when the spin head moving board 1312 of the spin drive device 1300 descends and interferes with the dispensing tip removal board 1461, causing the side projection 1461a to collide, the dispensing tip removal board 1461 moves downward from the fixed pump head PH and collides with the dispensing tip 71 or 72, applying downward pressure to the dispensing tip 71 or 72.

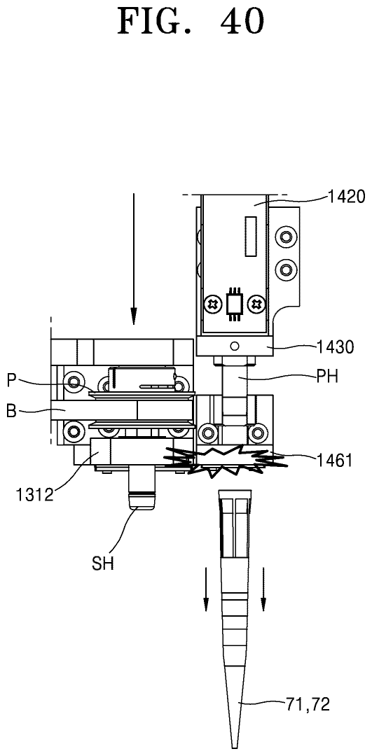

Subsequently, as shown in FIG. 40, when the spin head moving board 1312 of the spin drive device 1300 further descends while in contact with the side projection 1461a, the dispensing tip removal board 1461 moves further downward from the fixed pump head PH, thereby separating the dispensing tip 71 or 72 from the pump head PH and causing it to drop downward. Afterward, the dispensing tip removal board 1461 returns to its original position due to the elastic restoring force of the elastic spring 1462 when the dispensing tip 71 or 72 is removed and the spin head moving board 1312 moves upward.

Therefore, according to the present invention, it is possible to perform an operation of spinning the complex sample collector 30 with the spin head SH coupled to the complex sample collector 30, as well as an intricate and three-dimensional operation involving inserting the magnetic bar 82 into the stirring tip body 81 and moving it upward and downward to allow the spin head SH to rotate the stirring tip body 81 while simultaneously stirring magnetic beads B2, optimization of the design, such as reducing the number of threaded rods and simplifying components using the drive nut 1322, is possible, thereby lowering the production cost, and various functionalities and performances may be achieved, such as automatically removing the dispensing tips 71 and 72 using neighboring components such as the spin head moving board 1312 or the like.

In addition, in the present invention, the cartridge seating device 1200 is arranged in the Y-axis direction and the spin drive device 1300 and the pump drive device 1400 are raised and lowered in the Z-axis direction. Although the drawings illustrate the pretreatment of a single cartridge 100, it is also possible to arrange multiple cartridge seating devices 1200 in the X-axis direction, configure multiple spin drive devices 1300 and pump drive devices 1400, or enable movement along the X-axis direction to perform pretreatment on multiple cartridges 100.

While the present invention has been particularly shown and described with reference to embodiments thereof, it will be understood by those of ordinary skill in the art that various changes in form and details may be made therein without departing from the spirit and scope of the present invention as defined by the following claims. Therefore, the scope of the present invention should be defined only by the appended claims.

1. A complex sample pretreatment apparatus comprising:
a housing;
a cartridge seating device which is installed inside the housing and onto which a cartridge is seated;
a spin drive device which is installed in the housing, is coupled to an object to be spun which is mounted on the cartridge or mounted on the cartridge mounting device, and rotates the object to be spun; and
a pump drive device which is installed in the housing, is coupled to a dispensing tip mounted on the cartridge or on the cartridge mounting device, and dispenses a sample or reagent by using the dispensing tip.

2. The complex sample pretreatment apparatus of claim 1, wherein the cartridge seating device comprises:
a cartridge mounting board on which the cartridge is mounted; and
a mounting board moving device configured to move the cartridge mounting board in a first direction so that the cartridge mounted on the cartridge mounting board is positioned at a location corresponding to the spin drive device or the pump drive device.

3. The complex sample pretreatment apparatus of claim 2, wherein the mounting board moving device comprises:
a driven pulley freely rotatably installed on one side of the housing or frame;
a drive pulley freely rotatably installed on the other side of the housing or frame;
a mounting board drive motor configured to rotate the drive pulley; and
a belt fixed at one side to the cartridge mounting board, wound between the driven pulley and the drive pulley, and configured to move along a track.

4. The complex sample pretreatment apparatus of claim 2, wherein the cartridge seating device further comprises a heater device installed on the cartridge mounting board and configured to heat the cartridge.

5. The complex sample pretreatment apparatus of claim 4, wherein the heater device comprises:
a heating block installed on the cartridge mounting board and thermally contacting with at least a portion of the cartridge;
a heater configured to heat the heating block;
a heat-resistant sponge installed between the heater and the cartridge mounting board; and
a fastener configured to elastically couple the heating block to the cartridge mounting board using the heat-resistant sponge.

6. The complex sample pretreatment apparatus of claim 1, wherein the spin drive device comprises:
a spin head drive device which comprises a spin head to be coupled to a complex sample collector or a stirring tip body of the cartridge and moves the spin head up and down or spins the spin head; and
a magnetic bar drive device which comprises a magnetic bar, which is formed to pass through the spin head and is inserted into the stirring tip body, when the stirring tip body is coupled to the spin head, to generate magnetic force to allow magnetic beads in the cartridge to stir a sample, and moves the magnetic bar up and down.

7. The complex sample pretreatment apparatus of claim 6, wherein the spin head drive device comprises:
the spin head formed in a shape corresponding to the complex sample collector or the stirring tip body;
a spin head moving board configured to freely support the spin head;
a spin rotation motor installed on the spin head moving board and connected to a rotation axis of the spin head to spin the spin head;
a head lifting and lowering threaded rod which passes through the spin head moving board to allow the spin head moving board to move up and down and is freely installed on the housing or a frame installed in the housing; and
a head lifting and lowering motor configured to rotate the head lifting and lowering threaded rod.

8. The complex sample pretreatment apparatus of claim 7, wherein the magnetic bar drive device comprises:
the magnetic bar configured to pass through the spin head and allow the magnetic beads to move;
a magnetic bar moving board configured to support the magnetic bar;
a drive nut freely rotatably installed on the magnetic bar moving board, screwed with the head lifting and lowering threaded rod, and capable of screw-driven vertical movement;
a nut rotation motor which is installed on the magnetic bar moving board to enable the magnetic bar moving board to move up and down and is connected to the drive nut to rotate the drive nut; and
a moving board linear guide configured to guide lifting and lowering paths of the magnetic bar moving board and the spin head moving board to allow the lifting and lowering path of the magnetic bar moving board to coincide with the lifting and lowering path of the spin head moving board.

9. The complex sample pretreatment apparatus of claim 1, wherein the pump drive device comprises:
a pump head formed in a shape corresponding to the dispensing tip;
a dispensing pump connected to the pump head;
a pump moving board configured to support the dispensing pump;
a pump lifting and lowering threaded rod which passes through the pump moving board to allow the pump moving board to move up and down and is freely rotatably installed on the housing or a frame installed in the housing;
a pump lifting and lowering motor configured to rotate the pump lifting and lowering threaded rod; and
a dispensing tip removal device which interferes with the spin drive device and removes the dispensing tip from the pump head through the spin drive device.

10. The complex sample pretreatment apparatus of claim 9, wherein the dispensing tip removal device comprises:
a dispensing tip removal board which comprises a side projection formed to interfere with the spin drive device, is shaped to surround the pump head to separate the dispensing tip from the pump head as the dispensing tip removal board moves downward, and is formed to be movable up and down on the pump moving board;
an elastic spring configured to provide an elastic restoring force in an upward direction of the dispensing tip removal board; and
a removal board linear guide configured to guide lifting and lowering paths of the pump moving board and the dispensing tip removal board to allow the lifting and lowering path of the pump moving board to coincide with the lifting and lowering path of the dispensing tip removal board.

11. The complex sample pretreatment apparatus of claim 1, further comprising at least one of the following:
an air conditioning device which is installed in the housing and includes at least one of a ventilation fan, a deodorization device, an air purification device, or a filter, or a combination thereof; and
an ultraviolet sterilization device installed in the housing and configured to sterilize the cartridge or the cartridge seating device after pretreatment.
